SEQUENCE LISTING (25 sequences)

SEQ ID NO: 1 - DNA, 201 bp, Homo sapiens
  misc_feature (101)..(101): n is the major allele g or minor allele t
gatttatctgtattcctaaatcctccaggtcaaaatcgtagaacctctgtgtttctctcttcttcattctttagacaatatatagttaataaagttatcanccttttttcagaatcttttagtatcttctgtttcctggatcagatccttattatttcgtatctgagccatacaccagcctcttaaagagcctccgtaaat

SEQ ID NO: 2 - DNA, 201 bp, Homo sapiens
  misc_feature (101)..(101): n is major allele g or minor allele a
atgccacattttctgaatccagtctatcattgagggacatttggattggttccaagcctttgctattgtgaatagtgccgcagtaaacatacatgtgcatntgtctttacagaagcatgatttataatcctttgggtatatacccagtaatgggatgactgagtcaaatgctatttctagttcttgaggaaccaccacact

SEQ ID NO: 3 - DNA, 201 bp, Homo sapiens
  misc_feature (101)..(101): n is major allele t or minor allele c
cagtgggatttctcattctactttatagaatgaagggcttccctgatgaatggtggataaaaggcaattaaatttataactaaatatgttgtaattttgtnttctgacaagctaaattagcattatatagtccaatcaaagtgcccagagagagccaggtcactgaaacaagattttactaatgtctctgcctgcaaggcc

SEQ ID NO: 4 - DNA, 201 bp, Homo sapiens
  misc_feature (101)..(101): n is major allele c or minor allele t
agggtataggaaataccttaaggagagtgtacaagataaaaattcagttgtttggcttagagataaaaaggagatgatttttgtgttattgtatgtaagtncttcttactaggctgctgagtatggttatgcaagttgtgcactaaacagccatacatagcaatctgcttttttcttttcaattcactgtctacttagtca

SEQ ID NO: 5 - DNA, 201 bp, Homo sapiens
  misc_feature (101)..(101): n is major allele g or minor allele a
gaatagcaatgatttttactacttaaaaaataataaactcccaggcaagaaaccacccttattttgttcctgattttgttcacgtgggaaaacatgagttntgtaatgctcactacacaggggggcgctgtgtagcgcccttcccttccatcttggttggtggatgcctgtgtttgagaagcagcagattccccctaggaa

SEQ ID NO: 6 - DNA, 201 bp, Homo sapiens
  misc_feature (101)..(101): n is major allele c or minor allele a
ataatgtgaataataatgtgaatctgtagagttaatacaatgtgctcactctgagctaaactaaggactgtatgtacattatcccacctaatcctcataanagcctccggaggtaggaacattaaatacctgagatcaatgaggtacccagtgagtggtaactagtattacactctttaataccaataattcccaaggaag

SEQ ID NO: 7 - DNA, 201 bp, Homo sapiens
  misc_feature (101)..(101): n is major allele t or minor allele a
ctttttatatggctgcatagtattccacggtgtatatatgccacattttctgaatccagtctatcattgagggacatttggattggttccaagcctttgcnattgtgaatagtgccgcagtaaacatacatgtgcatgtgtctttacagaagcatgatttataatcctttgggtatatacccagtaatgggatgactgagt

SEQ ID NO: 8 - DNA, 201 bp, Homo sapiens
  misc_feature (101)..(101): n is major allele c or minor allele t
aaattagctgggcgtggtggcaggctcctgtaatcccagctacctgggagcctgaggcaagagaatcacttgaacccaggaggcagaggttgctatgagcngagatcgtaccgctgcactccaccctgggtgacatagcaaaactctgtctcaaaaagaaaaaaaaaaaaaaaaaaaagggagctagaagcgtagctgtag

SEQ ID NO: 9 - DNA, 201 bp, Homo sapiens
  misc_feature (101)..(101): n is major allele a or minor allele c
tgagggctacagagtctctgcactaactgctaatctcccacttggaacaatcactacagcagaggaataaagccaacactgaagccgttaaaccttgcatngtttcttgtgtgttgactcttggacttcaagggtgtatggtacttctcagctgaatcagtcagggtgggctagggtgcttgtattagtcagggttttcta

SEQ ID NO: 10 - DNA, 201 bp, Homo sapiens
  misc_feature (101)..(101): n is major allele t or minor allele a
ttggtacataacagtactttttacattcattcacattactaagtacaactatttttcaaacgtttcttcaaccaggacataacagaaatgccaacaagacntgtaatccagtcgactctgtaaggatgcccataagcttgagagtagtgcctgacacataagaggatgcttaataagtgataataaataaatgaagatttt

SEQ ID NO: 11 - DNA, 201 bp, Homo sapiens
  misc_feature (101)..(101): n is major allele c or minor allele t
tgcgcctgtagcttctggcggtgtcggccaagttcgaggggaagccgctgcttcagagacaccgcctggtgaacgcgtgcctagcagaagcgctcctgcgnatccatgcctttgaacagaaaaccctaatcccagagcagtgggcccgtgagcggcagaaatgagggactgggacctgcacagccattaaattacaaatct

SEQ ID NO: 12 - DNA, 201 bp, Homo sapiens
  misc_feature (101)..(101): n is major allele a or minor allele t
tactaaaaatacaaaaattagctgggtgtgatggtgcatggctgtaaccccagctactcgggaggctgaggcaggagaatcacttgaacctaggaggcggnggttgcagtgagccgaaatcgcgccactgcacttcagcttggcgacggagcgagactccatctcaaaaaacaaaaaacaaaaaaaaaaaaaccacaaaca

SEQ ID NO: 13 - DNA, 201 bp, Homo sapiens
  misc_feature (101)..(101): n is major allele c or minor allele g
atggatgacaaagtagtgggaatctgaaatgcctacagtagaagtaagggtgtctttatattgggaatatcctcgatcccagggcccatttctaattcccncttctctctttccctgatacaggctatttttgaacgagaaggactttggctcaaatcaacagtttggtttcttactgtttacatctatgagtcaaagtag

SEQ ID NO: 14 - DNA, 201 bp, Homo sapiens
  misc_feature (101)..(101): n is major allele c or minor allele t
ttgaactcctggcctcaagcgatccacccgcctcggcctcccaaagtggttttggtgattatttgattaatgccggtcttgcccagtggaatggagggtcnctgcagacagggaccactttgctggccttttctcatagtgaagtctctttccttaccactgtatccttagtgtcacacataataggtgctcattaaatat

SEQ ID NO: 15 - DNA, 201 bp, Homo sapiens
  misc_feature (101)..(101): n is major allele c or minor allele t
ggagagagtaggcactgaataaatgcttgctggctggaatggagtgagaaagggcatcactgttgacttaccttcctgtgtcttccctttcagcggcccanatgtgcagcaccctggagcataactgtgcccacttctgcatcaacatccctggctcatacgtctgcaggtgcaaacaaggctacattctcaactcggatc

SEQ ID NO: 16 - DNA, 201 bp, Homo sapiens
  misc_feature (101)..(101): n is major allele a or minor allele t
acacttaccccagtagtggcagtgtggcagactgcacatcattgggagcagggctgtggaatgtgctcatgttgcatgcacacatgtgtgccagcaggggnggggaggcgaggtctacttgcacttgcatgccctggaaaaacagtgggaaggctgtaggtgagtgcctgctagcagagcaggagcgggaggatgctgtgt

SEQ ID NO: 17 - DNA, 201 bp, Homo sapiens
  misc_feature (101)..(101): n is major allele c or minor allele t
ggagggagagagggaggaaatgggggaggcaggtaggaggaagtttccggtttgcccaaggccacagagaaggaggcctggagcccttgtcctcccaggtnggggagcatcctggttcctttgttgaactcactctctcaggacccaagagcagccgtggatggaagaaaatgaaaagacaaaacgcaagtcctaagcaca

SEQ ID NO: 18 - DNA, 201 bp, Homo sapiens
  misc_feature (101)..(101): n is major allele g or minor allele A
gcctaaaatgagaccctcaatcaactgccttagttctctggacttcctcactgaagatttctggtctgggattgtagaaggacagtggaagggtgtggaantatgagagaggaaggtaaagggtgtgattgtacgcatgcatatgtgtgtgtgtgtctatgcataaccacagaccacagatgggggaatggatggacagtt

SEQ ID NO: 19 - DNA, 201 bp, Homo sapiens
  misc_feature (101)..(101): n is major allele g or minor allele a
gctctttattttgtggtttgacttctttcattcagcctaatgattctgagatttatccatgtttttgattgcatcagaggttgttccttggtattgctgantagcatcctattgtattggatataccacaatttatccattcatctgttgaagaacatttgggttgcttataggtttatggctgttataaataaagttgct

SEQ ID NO: 20 - DNA, 201 bp, Homo sapiens
  misc_feature (101)..(101): n is major allele a or minor allele c
ccccaggctgagagaagaaagaaggaagtcaaagtggccagaaccacccagatctaaccatccccaataataatagggatttaacagagcatgaaaaaaanccccaaaatcatgtttcagggtgtggaagtgggtacactgagaacagaggaaaactcacttctcccctcagaactgtgaatgagaaatgaagtcttctga

SEQ ID NO: 21 - DNA, 201 bp, Homo sapiens
  misc_feature (101)..(101): n is major allel t or minor allele c
tgcaggagtagctatacttagacaaaatagactttacatgaaaaaacaaagacaaagaaggtcattatataatgataaagggatcaattcagcaagagganataacaattgtaaatatatatgcatctaatactgtagcacccagatatataaaacaaatattagagctaaagagataaatgccaatacgataatagttgg

SEQ ID NO: 22 - DNA, 201 bp, Homo sapiens
  misc_feature (101)..(101): n is major allele c or minor allele a
aaagtaagtttctatttgcagataatatgataatgtatgctgaaaattctaaggaatttactaacaaactcctagaactaataagtgaatttagcaagatnacagaatacaaggcaaatatctaggaatctattgtatttgtatacaccagcaattaacaattagaaaatgaaatttcaaaattttatctacaatagcata

SEQ ID NO: 23 - DNA, 201 bp, Homo sapiens
  misc_feature (101)..(101): n is major allele g or minor allele c
ccaggttaccctgtggacaccgtggcataagcttcagtgttgtcgttctcctgggagaatagaggatagatatcccggaagagattgtgaagattgttctntagcccccacctaaagtcatttgtgaaatcacgttcaaatccaggaaatgctgaaattaaaaatgcaggtaagtaaaacagtagggctaagagagtggtt

SEQ ID NO: 24 - DNA, 201 bp, Homo sapiens
  misc_feature (101)..(101): n is major allele c or minor allele t
acgtgaaaagatgctcaacatcactagtcattagggaaatgtaaatcaaaatcacaatgaaattccagtgcacactcactaggatggttacaatcaaaganaataacacatattggcaaagatgtgaagaaactggaaccctcatacattgctggtaagaacgtaaaatggtgcaaaaacaccattaaaaaaaaaacacaa

SEQ ID NO: 25 - DNA, 201 bp, Homo sapiens
  misc_feature (101)..(101): n is major allele c or minor allele t
gaggaattggattaactgccttcacatgacaagacaggaaagaaaacatttttcagagttgcattttcttaagtgtttctgacccactgagaaactgttcntatttattcatctctcaaattcatagtgggcatttactagtgttgggtactgaggatacagaaatgaaaagatgggtaaggttttagccattgagatttt

(19) United States
(12) Patent Application Publication
(10) Pub. No.: US 2023/0357849 A1
(43) Pub. Date: Nov. 9, 2023

(54) GENETIC ALTERATIONS ASSOCIATED WITH EOSINOPHILIC ESOPHAGITIS AND METHODS OF USE THEREOF FOR THE DIAGNOSIS AND TREATMENT OF DISEASE

(71) Applicant: THE CHILDREN'S HOSPITAL OF PHILADELPHIA, Philadelphia, PA (US)

(72) Inventors: Hakon Hakonarson, Malvern, PA (US); Patrick Sleiman, Philadelphia, PA (US); Xiao Chang, Bryn Mawr, PA (US)

(73) Assignee: THE CHILDREN'S HOSPITAL OF PHILADELPHIA, Philadelphia, PA (US)

(21) Appl. No.: 18/044,471

(22) PCT Filed: Sep. 8, 2021
(86) PCT No.: PCT/US2021/049508

§ 371 Date: Mar. 8, 2023

Related U.S. Application Data
(60) Provisional application No. 63/075,791, filed on Sep. 8, 2020.

(51) Int. Cl. C12Q 1/6883; G01N 33/68

(52) U.S. Cl. CPC C12Q 1/6883; G01N 33/6869; C12Q 2600/156; C12Q 2600/136; G01N 2333/5409; G01N 2333/5437; G01N 2500/10; C12Q 2600/158

(57) ABSTRACT

Compositions and methods for the treatment and diagnosis of eosinophilic esophagitis are disclosed.

### CROSS-REFERENCE TO RELATED APPLICATION

This invention claims priority to U.S. Provisional Application No. 63/075,791 filed Sep. 8, 2020, the entire disclosure being incorporated herein by reference as though set forth in full.

This invention was made with government support under Grant No. HG006830 awarded by the National Institutes of Health. The government has certain rights in the invention.

### INCORPORATION-BY-REFERENCE OF MATERIAL SUBMITTED IN ELECTRONIC FORM

Incorporated herein by reference in its entirety is the sequence listing submitted via EFS-Web as a text file named SEQLIST.txt, created Sep. 8, 2021, and having a size of 12,393 bytes.

### FIELD OF THE INVENTION

This invention relates to the fields of allergic disorders and genome wide analysis studies which facilitate identification of genetic alterations associated with such disorders. More specifically, the invention provides a new panel of genetic markers associated with eosinophilic esophagitis and methods of use thereof in diagnosis and screening assays for the identification of efficacious therapeutic agents.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

Eosinophilic esophagitis (EoE) is an inflammatory disorder of the esophagus histologically characterized by accumulation of eosinophils in the esophageal epithelium. EoE has become a major cause of upper gastrointestinal morbidity, as the incidence of EoE has increased exponentially over the past two decades with estimates ranging from 1/2000 to 1/1000 persons in the US. Clinical symptoms of EoE include dysphagia, failure to thrive, vomiting and epigastric or chest pain. A diagnosis of EoE is made following endoscopy and biopsy upon finding isolated eosinophils in the esophagus having ruled out gastroesophageal reflux. Multiple reports indicate a gender bias, with males predominantly affected with a male-to-female ratio approaching 3:1. The rate of co-existing atopic disease in other organs is high, with up to 70% of subjects presenting with asthma or atopic dermatitis. It is known that genetic factors and their interaction with environmental exposures contribute to the risk of EoE. For example, twin studies reveal a strong heritability of EoE, and multiple genetic risk loci have been reported by both candidate-gene studies and genome-wide association studies (GWAS).

EoE is considered a food allergy-related disorder based on the high rate of food allergen sensitization and a higher rate of food anaphylaxis in cases compared with the general population. Furthermore, the majority of EoE cases undergo disease remission following introduction of an elemental formula diet that lacks allergens. Experimental modeling of EoE in mice has demonstrated a key role for adaptive immunity and Th2-cell cytokines (especially IL-5 and IL-13) in the disease process and a strong connection between allergic sensitization and inflammation in the respiratory tract and skin. EoE is inherited as a complex trait suggesting it is caused by multiple genetic variations interacting with environmental influences. Clearly, a need exists in the art for improved methods for diagnosis and management of this disorder.

### SUMMARY OF THE INVENTION

In accordance with the present invention, a method for detecting a propensity for developing eosinophilic esophagitis (EoE) in a subject in need thereof is provided. An exemplary method comprises detecting the presence of at least one genetic alteration in a target gene identified in said subject wherein if said genetic alteration is present, said patient has an increased risk for developing eosinophilic esophagitis, wherein said genetic alteration is present in a gene sequence from one or more loci of TMEM182, RAD50, SOX4, MATN2, PRKG1, RHOG, SHANK2, GPR12, RORA, SMAD3, GALNT1, CPNE4, URGCP, NAMPT, JAK2, and/or CCNY. The present inventors have discovered that such loci comprise single nucleotide polymorphisms (SNP) that indicate that the genetic alteration is present. In certain embodiments, the step of detecting the presence of said SNP comprises performing a process selected from the group consisting of detection of specific hybridization, measurement of allele size, restriction fragment length polymorphism analysis, allele-specific hybridization analysis, single base primer extension reaction, and sequencing of an amplified polynucleotide. In certain embodiments, an additional genetic alteration is present in a gene sequence from one or more loci of CAPN14, TSLP/WDR36, EMSY, and/or CLEC16A. In an additional embodiment, the genetic alteration is a sex-specific genetic alteration. In some embodiments, the sex-specific alteration may be selected from TMEM182, CPNE4, and/or URGCP or NAMPT, JAK2, and/or CCNY. In certain embodiments, the subject may also be suffering from at least one additional disease selected from asthma, allergies, atopic dermatitis, celiac disease, selective IgA deficiency, Systemic lupus erythematosus, multiple sclerosis, rheumatoid arthritis, inflammatory bowel disease, Chron's disease, ulcerative colitis, and/or type 1 diabetes. Kits for practicing the method described above are also within the scope of the invention. Methods for treating EoE and other related disorders are also disclosed.

In another aspect, the invention provides a method for identifying agents which modulate the development or progression of eosinophilic esophagitis. An exemplary method entails providing cells expressing at least one nucleic acid comprising a genetic alteration associated with EoE as described above, providing cells which express the cognate wild type sequence lacking the genetic alterations, contacting each cell type with a test agent and analyzing whether the agent alters a cellular parameter associated with the presence of eosinophilic esophagitis in the cells of step a) relative to those of step b), thereby identifying agents which alter said parameter. Such parameters include without limitation, increased expression of IL-5 or IL-13, epidermis development, epithelial cell differentiation, serine protease inhibition, altered cell cycle progression, or division, microtubule disruption, histone acetylation, DNA methylation, chromosomal segregation, ubiquitin conjugation, and phosphoinositide mediated signaling, and altered mitosis.

In particularly preferred embodiments, the agent alters mRNA or protein levels of the EoE associated genes of the invention, i.e., TMEM182, RAD50, SOX4, MATN2, PRKG1, RHOG, SHANK2, GPR12, RORA, SMAD3, GALNT1, CPNE4, URGCP, NAMPT, JAK2, and/or CCNY. Preferably, the alterations in expression levels are observed in blood or esophageal cells. Also provided are kits for practicing the screening method described above.

### BRIEF DESCRIPTION OF THE DRAWINGS

FIGS. 1A -1D: Population structures of the study samples from four cohorts revealed by principle component analysis.

FIGS. 2A-2L: Scree plot of the first 10 PCs. A scree plot was generated by plotting the percentage of variances explained (eigenvalue) by the first 10 PCs against the number of PCs. Based on this, the optimal number of PCs (where the “elbow” point occurred) was selected. The first five, two, two, four, six, three, four, four, four, three, four and four PCs were selected for subfigures A, B, C, D, E, F, G, H, I, J, K, L respectively

FIGS. 3A-3C: Quantile-quantile plots show −log10(p-value) of observed genome-wide association results against expected association results for EoE. Genomic inflation factors are 1.03 for the association results of meta-analysis (FIG. 3A), 1.03 for the association results of meta-analysis from males (FIG. 3B), 1 for the association results of meta-analysis from females (FIG. 3C).

FIGS. 4A-4C: Manhattan plot of the GWAS meta-analysis of EoE and sex-specific analysis. Known loci, novel common loci and novel low-frequency loci are colored in blue, yellow and red respectively. (FIG. 4A) meta-analysis of all samples, (FIG. 4B) meta-analysis of males, (FIG. 4C) meta-analysis of females.

FIGS. 5A-5K: Regional plot of the genome-wide significant loci associated with EoE using LocusZoom. Purple diamond indicates the most significantly associated SNP, and circles represent the other SNPs in the region, with coloring from blue to red corresponding to r2 values from 0 to 1 with the index SNP. * denotes low-frequency loci.

FIGS. 6A-6E: Regional plot of the sex-specific genome-wide significant loci associated with EoE using LocusZoom. Purple diamond indicates the most significantly associated SNP, and circles represent the other SNPs in the region, with coloring from blue to red corresponding to r2 values from 0 to 1 with the index SNP. * denotes low-frequency loci. ♂ denotes male-specific loci. ♀ denotes female-specific loci.

FIG. 7: Genetic correlations between EoE and other phenotypes. *: P-value<0.05, **: P-value<0.01, ***: P-value<0.001.

FIGS. 8A-8F: Boxplots of the gene expression levels and genotypes of the top SNP at loci 2q12.1 (FIG. 8A), 5q31.1 (FIG. 8B), 11p15.4 (FIG. 8C), 15q22.2 (FIG. 8D), 15q23(FIG. 8E) and 9p24.1 (FIG. 8F) in Esophagus tissues. ♀ denotes female-specific loci.

FIGS. 9A-9B: Enriched gene sets and pathways identified by FUMA web server.

FIGS. 10A-10C: Sequences comprising the SNPs of the present invention.

### DETAILED DESCRIPTION OF THE INVENTION

Eosinophilic esophagitis (EoE) is an allergic disorder characterized by infiltration of the esophagus with eosinophils. We had previously reported association of the 5q22.1 (TSLP) locus with EoE. Additionally, association of EoE with 2p23.1 (CAPN14), and 11q13.5 (EMSY/LRRC32), has been reported. Recently, a new locus at 16p13.13 (CLEC16A), a locus previously identified in Asthma and Type 1 diabetes, has been identified. Given that EoE is highly heritable, the reported loci so far only explain a small fraction of its heritability suggesting that additional risk loci remain uncovered.

Here, we report the association of eleven novel loci with EoE. These include TMEM182, that encodes transmembrane protein 182, RAD50 that is involved in DNA double-strand break repair, SOX4 that is involved in the regulation of embryonic development and the determination of the cell fate, MATN2 that is involved in the formation of filamentous networks in the extracellular matrices of various tissues, PRKG1 that is involved in mediation of the nitric oxide/cGMP signaling pathway, RHOG which encodes a small GTPase that is involved in signaling transduction cascades, SHANK2 that encodes proteins that function as molecular scaffolds in the postsynaptic density of excitatory synapses, GPR12 that was previously associated with gastro-esophageal reflux and eosinophil count, RORA which binds to hormone response elements upstream of several genes to enhance their expression, SMAD3 which is involved in regulating gene activity and cell proliferation, and GALNT1 which catalyzes the transfer of GalNAc to serine and theonine residues on target proteins.

Additionally, we report the association of novel sex-specific loci associated with EoE, including TMEM1482, CPNE4 that is involved in membrane trafficking, mitogenesis and development, URGCP which promotes hepatocellular growth and survival, NAMPT that is involved in metabolism, stress response, and aging, JAK2 which plays a role in the pathogenesis through the JAK-STAT signaling pathway, and CCNY that is involved in controlling cell division cycles.

The following definitions are provided to facilitate an understanding of the present invention.

### I. Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “sex-specific genetic alteration” or “sex-specific alteration” refers to a genetic alteration that is associated with an altered risk of developing a disease when found in a specific sex. Each sex-specific genetic alteration is associated with an altered risk of developing the disease in either a male or a female patient. In certain embodiments, a sex-specific genetic alteration is not associated with an altered risk of developing the disease in the unspecified sex. In another embodiment, a sex-specific genetic alteration is more heavily associated with an altered risk of developing the disease in the specified sex but may be associated, to a lesser degree, with an altered risk of developing the disease in the unspecified sex. A gene that is associated with a sex-specific alteration is referred to as a “sex-specific gene” or a “sex-specific loci.”

The term “EoE-associated sex-specific genetic alteration” or “EoE-associated sex-specific alteration” refers to a sex-specific alteration that is associated with an altered risk of developing EoE.

A “single nucleotide polymorphism (SNP)” refers to a change in which a single base in the DNA differs from the usual base at that position. These single base changes are called

SNPs or “snips.” Millions of SNP's have been cataloged in the human genome. Some SNPs such as that which causes sickle cell are responsible for disease. Other SNPs are normal variations in the genome.

A “copy number variation (CNV)” refers to the number of copies of a particular gene or segment thereof in the genome of an individual. CNVs represent a major genetic component of human phenotypic diversity. Susceptibility to genetic disorders is known to be associated not only with single nucleotide polymorphisms (SNP), but also with structural and other genetic variations, including CNVs. A CNV represents a copy number change involving a DNA fragment that is ˜1 kilobases (kb) or larger (Feuk et al. 2006a). CNVs described herein do not include those variants that arise from the insertion/deletion of transposable elements (e.g., ˜6-kb KpnI repeats) to minimize the complexity of future CNV analyses. The term CNV therefore encompasses previously introduced terms such as large-scale copy number variants (LCVs; Iafrate et al. 2004), copy number polymorphisms (CNPs; Sebat et al. 2004), and intermediate-sized variants (ISVs; Tuzun et al. 2005), but not retroposon insertions. The terminology “duplication-containing CNV” is also used herein below consistent with the CNV definition provided.

“EoE-associated SNP” or “EoE-associated specific marker” or “EoE-associated informational sequence molecule” is a SNP or marker sequence which is associated with an altered risk of developing EoE not found normal in patients who do not have this disease. 25 Such markers may include but are not limited to nucleic acids, proteins encoded thereby, or other small molecules. Thus, the phrase “EoE-associated SNP containing nucleic acid” is encompassed by the above description.

A “sex-specific EoE-associated SNP” or “sex-specific EoE-associated specific marker” or “sex-specific EoE-associated informational sequence molecule” refers to EoE-associated SNP that is associated with an altered risk of developing EoE when found in a specific sex. Each sex-specific EoE-associated SNP is associated with an altered risk of developing EoE in either a male or a female patient. In certain embodiments, a sex-specific EoE-associated SNP is not associated with an altered risk of developing EoE in the unspecified sex. In another embodiment, a sex-specific EoE-associated SNP is more heavily associated with an altered risk of developing EoE in the specified sex,but may still be associated with an altered risk of developing EoE in the unspecified sex.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose. EoE associated nucleic acids may be affixed or immobilized to a solid matrix. Affixed or immobilized as used herein refers to a linkage that is stable in solution, such that the nucleic acids remain attached to the solid matrix under different processing or experimental conditions.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

The phrase “partial informative CNV” is used herein to refer to a nucleic acid that hybridizes to sequences comprising a duplication on a chromosome however, the partial informative CNV may not be identical to the duplication, rather, the CNV may correspond to only a portion of the duplication, but yet is still informative for the same.

“Target nucleic acid” as used herein refers to a previously defined region of a nucleic acid present in a complex nucleic acid mixture wherein the defined wild-type region contains at least one known nucleotide variation which may or may not be associated with EoE but is informative of the risk of EoE. The nucleic acid molecule may be isolated from a natural source by cDNA cloning or subtractive hybridization or synthesized manually. The nucleic acid molecule may be synthesized manually by the triester synthetic method or by using an automated DNA synthesizer. When cloning a target nucleic acid comprising a deletion, the skilled artisan is well aware of methods for selecting nucleic acids of a sufficient length flanking the affected region to facilitate cloning the region into a vector of choice.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10-6-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under predetermined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any EoE specific marker gene or nucleic acid, but does not hybridize to other nucleotides. Also polynucleotide which “specifically hybridizes” may hybridize only to an EoE-specific marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5″ C+16.6 Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57″ C. The Tₘ of a DNA duplex decreases by 1-1.5″ C with every 1% decrease in homology. Thus, targets with greater than about 75% sequence identity would be observed using a hybridization temperature of 42″ C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 25 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together. Vectors engineered to express nucleic acids encoding proteins having deletions can be generated by providing altered sequence along with flanking sequences of a sufficient length such that cloning into a vector is possible. Such flanking sequences can be between 10, 20, 50, 100, or 200 nucleotides in length.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the EoE specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the EoE specific marker nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism”, or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably an EoE specific marker molecule, such as a marker described hereinbelow. Samples may include but are not limited to cells, body fluids, including blood, serum, plasma, cerebral spinal fluid, urine, saliva, tears, pleural fluid and the like.

The terms “agent” and “compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the CNV or SNP-containing nucleic acids described herein or their encoded proteins. Agents and compounds may also be referred to as “test agents” or “test compounds” which are evaluated for potential biological activity by inclusion in screening assays described herein below.

The term “modulate” as used herein refers to increasing/promoting or decreasing/inhibiting a particular cellular, biological or signaling function associated with the normal activities of the genetic alteration containing molecules described herein or the proteins encoded thereby. For example, the term modulate refers to the ability of a test compound or test agent to interfere with signaling or activity of a gene or protein of the present invention.

### II. Methods of Using EoE-Associated SNPs for Diagnosing a Propensity for the Development of EoE

The present invention provides methods of diagnosing EoE in a patient or methods for identifying a patient having an increased risk of developing EoE. Diagnosis, as used herein, includes not only the initial identification of EoE associated with the genetic alterations described herein in a patient but confirmatory testing, or screening in patients who have previously been identified as having or likely to have EoE. The methods include the steps of providing a biological sample from the patient, measuring the amount of particular sets, or any or all of the EoE associated markers present in the biological sample, preferably a tissue and/or blood plasma sample, and determining if the patient has a greater likelihood of EoE based on the amount and/or type of EoE marker expression level determined relative to those expression levels identified in patient cohorts of known outcome. A patient has a greater likelihood of having EoE when the sample has a marker expression profile associated with patients previously diagnosed with EoE. The compositions and methods of the invention are useful for the prognosis and diagnosis and management of EoE

In another aspect, the patient sample may have been previously genotyped and thus the genetic expression profile in the sample may be available to the clinician. Accordingly, the method may entail storing reference EoE associated marker sequence information in a database, i.e., those SNPs statistically associated with a more favorable or less favorable prognosis as described herein, and performance of comparative genetic analysis on the computer, thereby identifying those patients having increased risk EoE.

EoE-related SNP-containing nucleic acids, including but not limited to those listed below may be used for a variety of purposes in accordance with the present invention. EoE-associated SNP-containing DNA, RNA, or fragments thereof may be used as probes to detect the presence of and/or expression of EoE specific markers. Methods in which EoE specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting EoE-associated SNPs may be conducted on any type of biological sample, including but not limited to body fluids (including blood, urine, serum, gastric lavage, cerebral spinal fluid), any type of cell (such as brain cells, white blood cells, mononuclear cells, fetal cells in maternal circulation) or body tissue.

Clearly, EoE-associated SNP-containing nucleic acids, vectors expressing the same, EoE SNP-containing marker proteins and anti-EoE specific marker antibodies of the invention can be used to detect EoE associated SNPs in body tissue, cells, or fluid, and alter EoE SNP-containing marker protein expression for purposes of assessing the genetic and protein interactions involved in the development of EoE.

In most embodiments for screening for EoE-associated SNPs, the EoE-associated SNP-containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Any of the aforementioned techniques may be used to detect or quantify EoE-associated SNP marker expression and accordingly, diagnose EoE.

### III. Kits and Articles of Manufacture

Any of the aforementioned products can be incorporated into a kit which may contain a EoE-associated SNP specific marker polynucleotide or one or more such markers immobilized on a Gene Chip, an oligonucleotide, a polypeptide, a peptide, an antibody, a detectable label, marker, reporter, a pharmaceutically acceptable carrier, a physiologically acceptable carrier, instructions for use, a container, a vessel for administration, an assay substrate, or any combination thereof. Immobilization on a solid support refers to methods for linking the nucleic acid molecules to the support such that they cannot be stripped from the support via washing.

### IV. Methods of Using EoE-Associated SNPs for the Development of Therapeutic Agents

Since the SNPs identified herein have been associated with the etiology of EoE, methods for identifying agents that modulate the activity of the genes and their encoded products containing such SNPs should result in the generation of efficacious therapeutic agents for the treatment of this disorder.

Several regions of the human genome such as those listed in Tables 2, 3, and 4 provide suitable targets for the rational design of therapeutic agents. Small nucleic acid molecules or peptide molecules corresponding to these regions may be used to advantage in the design of therapeutic agents that effectively modulate the activity of the encoded proteins.

Molecular modeling should facilitate the identification of specific organic molecules with capacity to bind to the active site of the proteins encoded by the SNP-containing nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered EoE associated gene. These host cell lines or cells are defective at the polypeptide level. The host cell lines or cells are grown in the presence of drug compound. Biological functions associated with the altered EoE genes are then measured to determine if the compound is capable of regulating these functions in the defective cells. Host cells contemplated for use in the present invention include but are not limited to bacterial cells, fungal cells, insect cells, mammalian cells, and plant cells. However, mammalian cells, particularly esophageal cells are preferred. The EoE-associated SNP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIP5, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the EoE-associated SNP containing nucleic acids of the present invention or functional fragments thereof provide a system in which to screen potential compounds or agents for the ability to modulate the development of EoE. Thus, in one embodiment, the nucleic acid molecules of the invention may be used to create recombinant cell lines for use in assays to identify agents which modulate aspects of cellular metabolism associated with EoE and aberrant eosinophil function. Also provided herein are methods to screen for compounds capable of modulating the function of proteins encoded by SNP-containing nucleic acids.

Another approach entails the use of phage display libraries engineered to express fragment of the polypeptides encoded by the SNP-containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of SNP-containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of EoE-associated SNP-containing nucleic acids enables the production of strains of laboratory mice carrying the EoE-associated SNPs of the invention. Transgenic mice expressing the EoE-associated SNP of the invention provide a model system in which to examine the role of the protein encoded by the SNP-containing nucleic acid in the development and progression towards EoE. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of EoE-associated SNP-containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated EoE-associated SNP containing genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou- racil, (FIAU). By this counter selection, the number of homologous recombinants in the surviving transformants can be increased. Utilizing EoE-associated SNP-containing nucleic acid as a targeted insertional cassette provides means to detect a successful insertion as visualized, for example, by acquisition of immunoreactivity to an antibody immunologically specific for the polypeptide encoded by EoE-associated SNP nucleic acid and, therefore, facilitates screening/selection of ES cells with the desired genotype.

As used herein, a knock-in animal is one in which the endogenous murine gene, for example, has been replaced with human EoE-associated SNP-containing gene of the invention. Such knock-in animals provide an ideal model system for studying the development of EoE.

As used herein, the expression of a EoE-associated SNP-containing nucleic acid can be targeted in a “tissue specific manner” or “cell type specific manner” using a vector in which nucleic acid sequences encoding all or a portion of an EoE-associated SNP containing nucleic acid are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the EoE-associated SNP of the invention may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13 (6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; a NEGR1 promoter, a GRMS promoter, and a promotor of any gene listed in the tables below.

Methods of use for the transgenic mice of the invention are also provided herein. Transgenic mice into which a nucleic acid containing the EoE-associated SNP or its encoded protein have been introduced are useful, for example, to develop screening methods to screen therapeutic agents to identify those capable of modulating the development of EoE.

### V. Pharmaceutical and Peptide Therapies

The elucidation of the role played by the EoE associated SNP containing nucleic acids described herein facilitates the development of pharmaceutical compositions useful for treatment and diagnosis of EoE. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Several treatment regimens for the treatment of EoE are known. These include, without limitation, elimination and elemental diets to decrease allergen exposure, acid suppression to treat gastroesophageal reflux disease, which may mimic or contribute to eosinophilic esophagitis, topical glucocorticoids to decrease esophageal inflammation and esophageal dilation to treat strictures. Proton pump inhibitors (PPIs) are often used as first treatment for EoE. PPIs have been shows to reduce esophageal inflammation in patient with EoE by reducing acid production in the stomach. Although the mechanism of PPIs is thought to primarily involve acid blockade, PPIs are thought to affect EoE by means of other mechanisms. After treatment with PPIs, patients have shown a large decrease in the number of eosinophils and inflammation after treatment. Corticosteroids are also helpful in controlling the inflammation caused by EoE. In one aspect of the invention, a test and treatment method is disclosed wherein a patient is assessed for an EoE associated genetic alteration as disclosed herein and treating patients harboring such alterations with agents known to be useful for ameliorating symptoms associated with EoE.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The materials and methods set forth below are provided to facilitate the practice of the following examples.
Samples: This study included four independent EoE cohorts. The first cohort (CHOP-1 cohort) contained EoE patients of 766 European ancestry and 4962 matched controls. Most cases in this cohort was collected from five sites including the CHOP, University of California San Diego, Northwestern University, Stanford University, University of Colorado and Academic Medical Center (Amsterdam) [9]. Additional cases were accessed from the Electronic Medical Records and Genomics (eMERGE) Network.

The second cohort (CHOP-2 cohort) included 352 patients of European ancestry and 1,025 matched controls from CHOP, which was slightly enlarged from the previous cohort [9]. The third cohort (CHOP-3 cohort) included 279 EoE patients of European ancestry and 2475 matched controls recently recruited and genotyped on the Illumina GSA array at CHOP. Cases were biopsy proven with an eosinophils/hpf (400×) count of ≥24 on proton pump inhibitor (PPI) therapy for at least 8 weeks. Controls were defined as typically developing children aged 12 years and older with no ICD9/10 code for EoE or related conditions (Table 1), which were recruited through the CHOP Health Care Network during the same timeframe. Blood-derived DNA samples from participants of the three case cohorts were genotyped at the Center for Applied Genomics (CAG), at CHOP, using the Illumina HumanHap550/610, Illumina OmniExpress and Infinium Global Screening (GSA) SNP array, respectively.

TABLE 1
Codes for EoE or related conditions for subject
exclusion in controls of Third Cohort

| ICD9/ | | |
|---|---|---|
| 10 | Code | Name |
| ICD9 | 279.x | Disorders involving the immune mechanism |
| | 287 | Allergic purpura |
| | 288.1 | Functional disorders of polymorphonuclear neutrophils |
| | 372.14 | Other chronic allergic rhinoconjunctivitis |
| | 493.x | Asthma |
| | 477.x | Allergic rhinitis |
| | 691.8 | Eczema, atopic dermatitis |
| | 995.2- | Unspecified adverse effect of drug medicinal and |
| | 995.3 | biological substance not elsewhere classified; and |
| | | unspecified allergy |
| | V14.x | Personal Hx of allergy to medicinal agents |
| | V15.0x | Personal Hx of allergy, other than to medicinal agents |
| ICD10 | D69.0 | Allergic purpura |
| | D71 | Functional disorders of polymorphonuclear neutrophils |
| | D80 | Immunodeficiency with predominantly antibody |
| | | defects |
| | D81 | Combined immunodeficiencies |
| | D82 | Immunodeficiency associated with other major defects |
| | D83 | Common variable immunodeficiency |
| | D84 | Other immunodeficiencies |
| | D89 | Other disorders involving the immune mechanism, |
| | | not elsewhere classified |
| | H10.45 | Other chronic allergic conjunctivitis |
| | J30 | Vasomotor and allergic rhinitis |
| | J45 | Asthma |
| | L20 | Atopic dermatitis |
| | T50.905 | Adverse effect of unspecified drugs, medicaments and |
| | | biological substances |
| | T78.4 | Other and unspecified allergy |
| | Z88 | Allergy status to drugs, medicaments and biological |
| | | substances |
| | Z91.0 | Allergy status, other than to drugs and biological |
| | | substances |

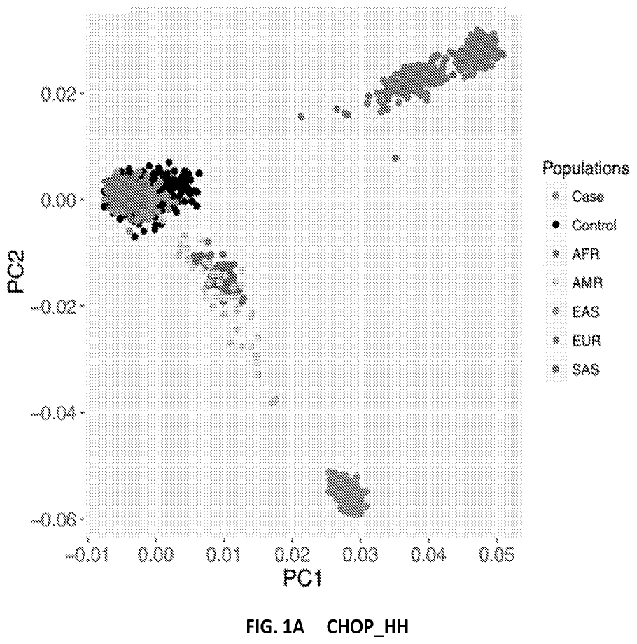

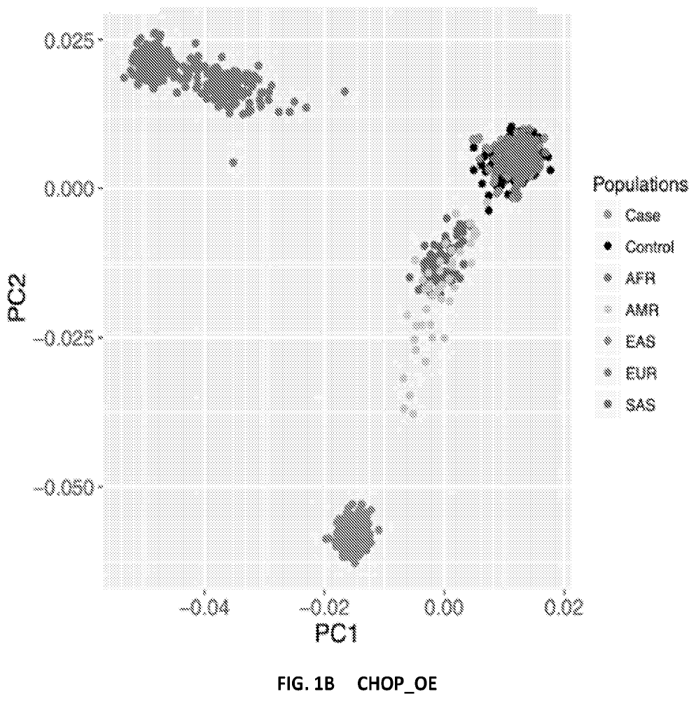

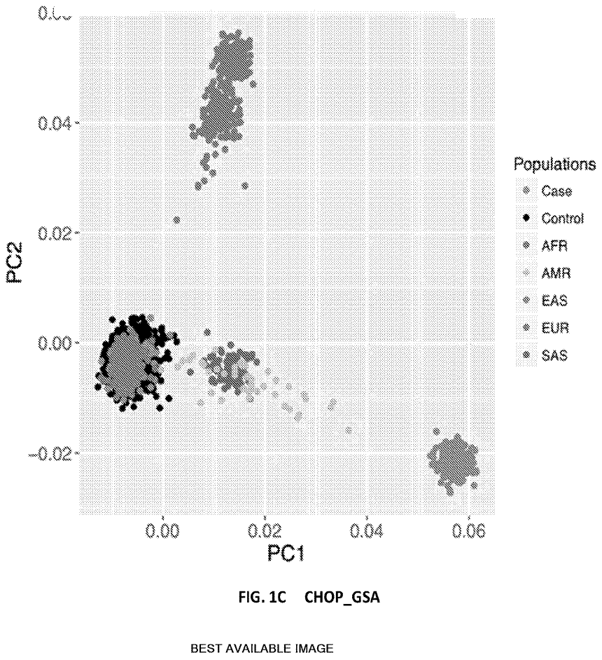

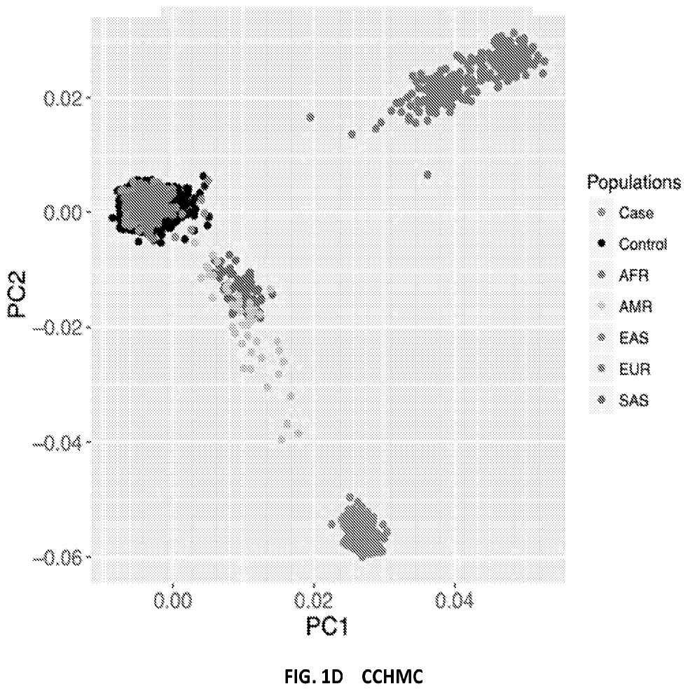

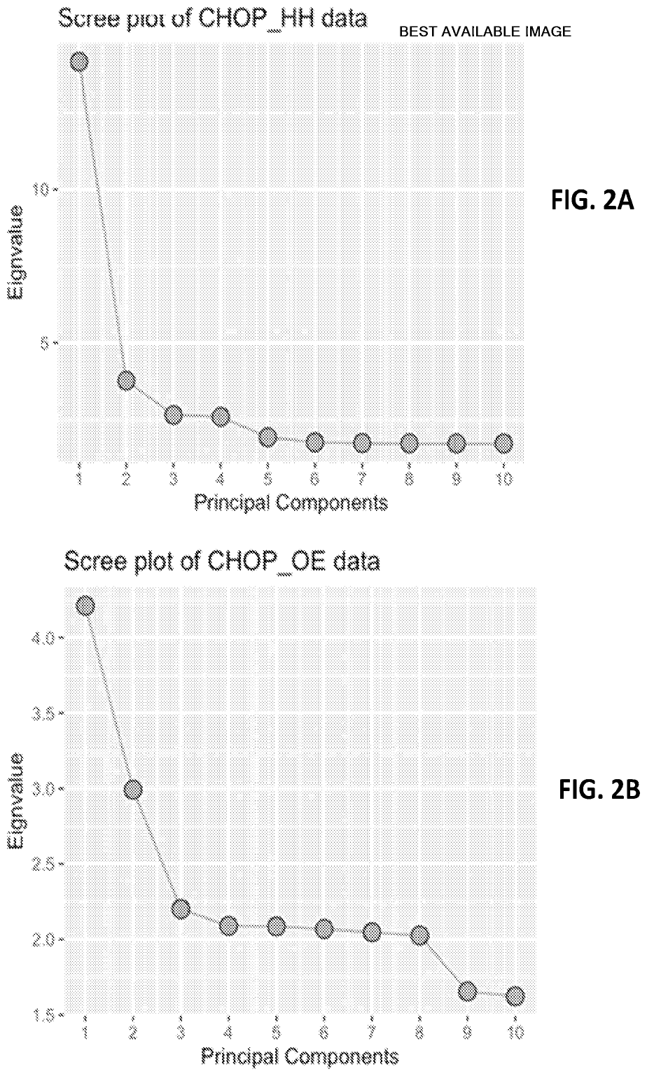

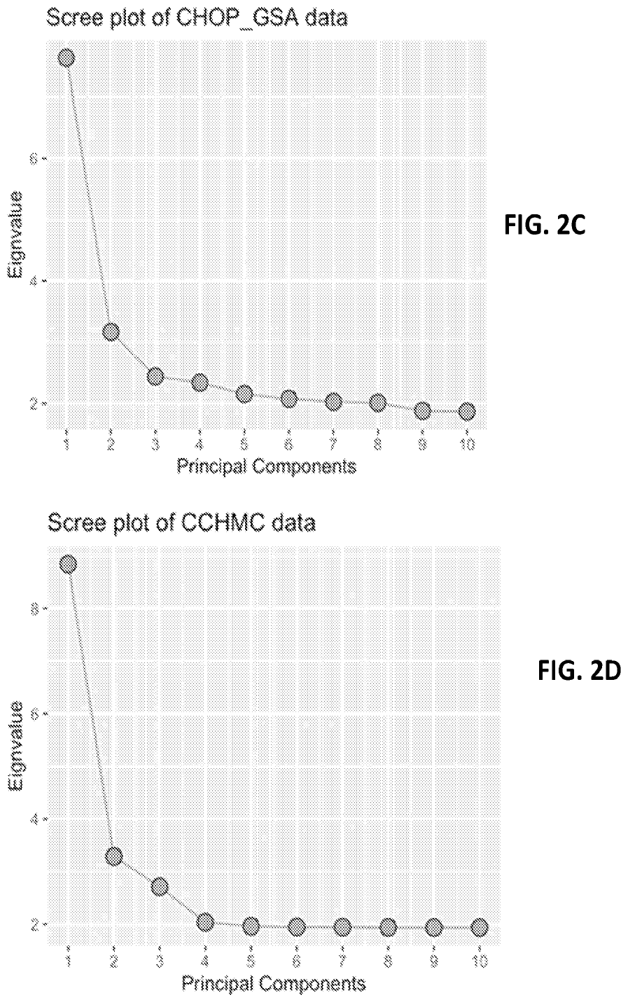

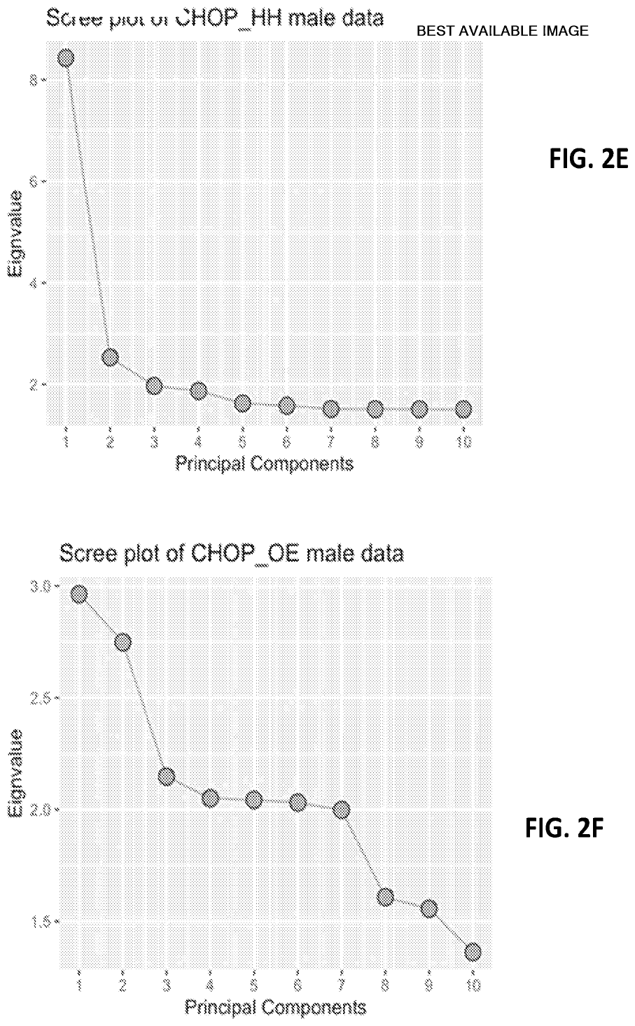

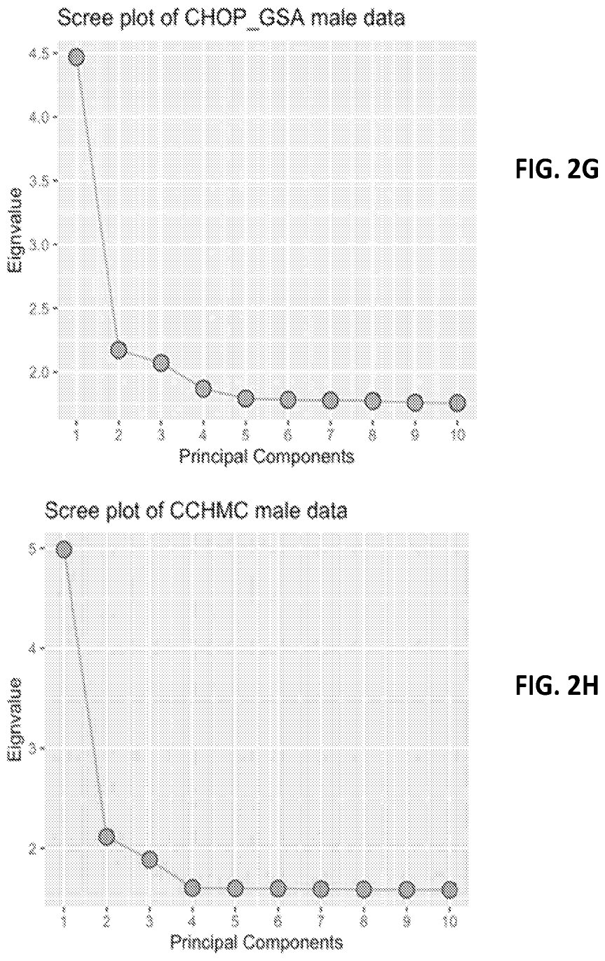

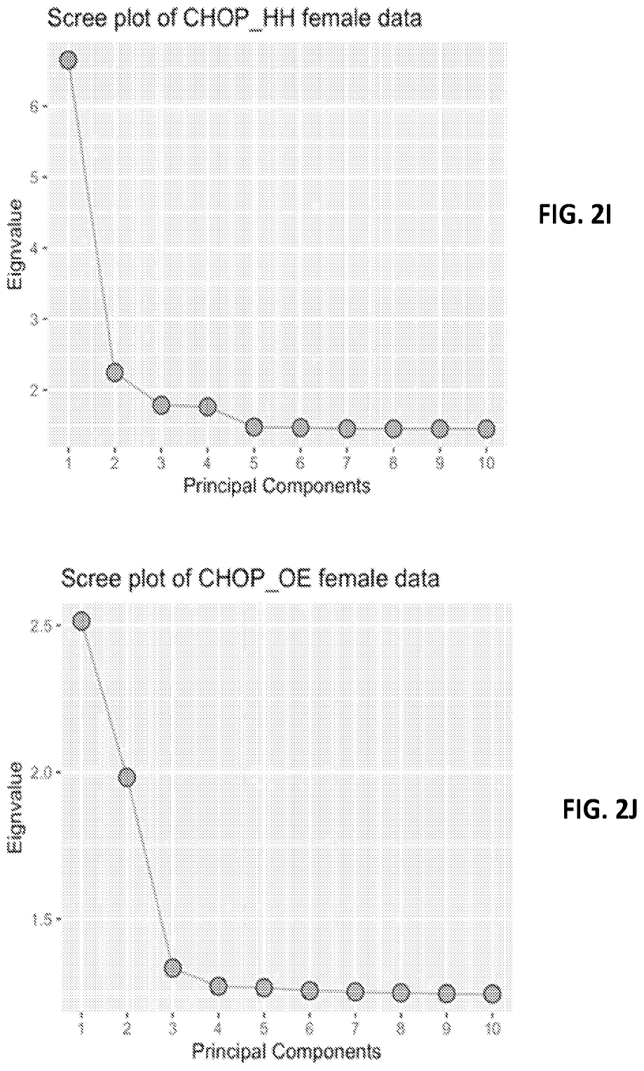

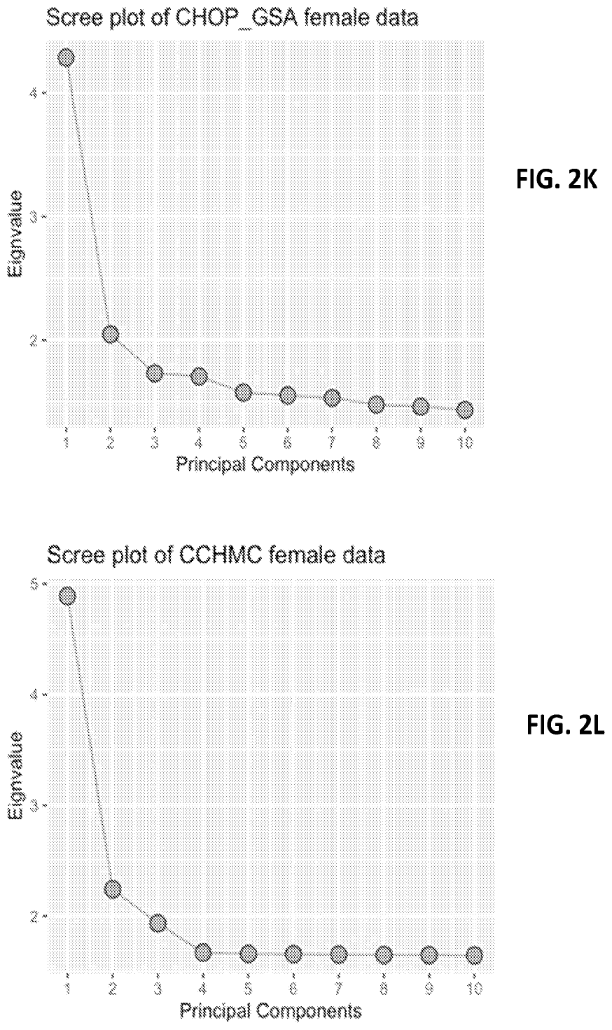

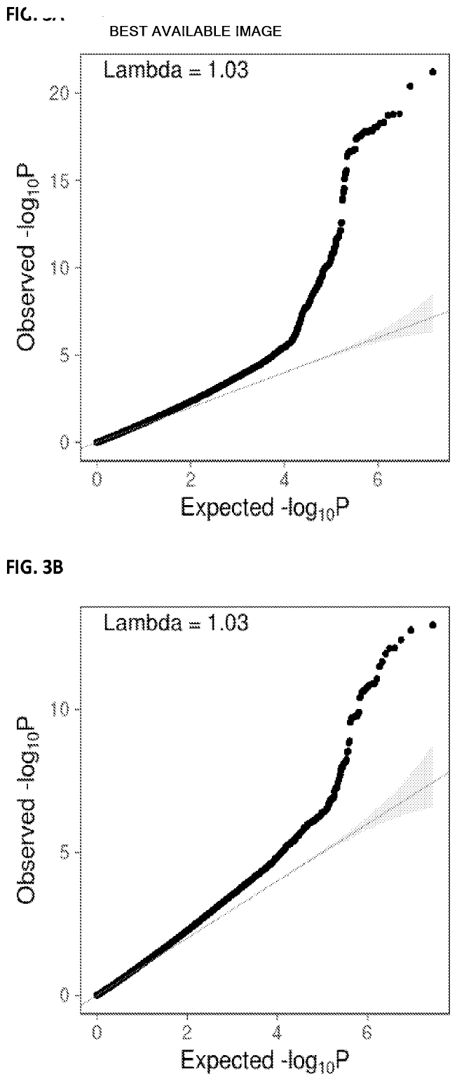

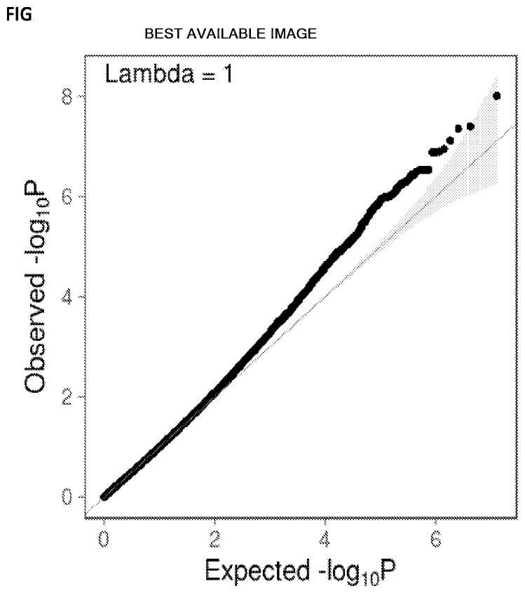

The fourth cohort (eMERGE cohort) consisted of 533 EoE patients of European ancestry and 5,172 matched controls. These EoE cases were collected from Cincinnati Children's Hospital Medical Center, and defined as peak eosinophil count ≥15 eosinophils/high-power field in esophageal biopsy sections, and verified by a physician to meet the diagnostic criteria for EoE [6]. Control subjects were composed of healthy self-reported European-American children of the Cincinnati Genomic Control Cohort and a non-EoE control cohort from the University of Michigan Health and Retirement Study. The genotyping data for 5 the CCHMC cohort was acquired from dbGAP (phs000494.v1.p1 and phs000428.v1.p1). Genotyping was performed on the Illumina OMNI-5 and OMNI-2.5 SNP arrays.
Population stratification and genome-wide imputation: EIGENSTRAT was used to detect potential substructures and outliers [10]. Participants with European ancestry were strictly selected by comparing principal component analysis results of participants and reference populations from Hapmap3 (FIG. 1A-1D). Samples with chip-wide genotyping failure rate greater than 5% were excluded. SNP markers with minor allele frequencies less than 1%, genotyping failure rates greater that 2%, and Hardy-Weinberg P-Values less than 1×10⁻⁶ were removed before genotype imputation. Pairwise identity-by-descent values were calculated by PLINK to remove cryptic relatedness and duplicated samples [11]. Genotype imputation was performed with the Michigan Imputation Server using minimac4 imputation algorithm [12]. The whole genome sequencing data from over 100,000 individuals derived from the Trans-Omics for Precision Medicine (TOPMed) program, were used as the imputation reference panel. Since the TOPMed imputation reference panel achieved a significant improvement in imputation qualities and accuracies of rare variants [13], besides common SNPs (MAFs>1%, Rsq (imputation quality metric)>0.3), low-frequency SNPs (MAFs between 0.1% and 1%) with high imputation confidence (Rsq >0.5) were also retained for association analysis.
Association Analysis: Association analyses were performed using logistic regression with an additive model on the imputed dosage of the effect allele while adjusting for sex and the informative PCs (FIG. 2A-2L). For the sex-specific GWAS, the association analysis was adjusted for the informative PCs. Meta-analysis was performed by GWAMA. Fixed-effects P values were reported. No genomic inflation was detected (FIG. 3A-3C).
Genetic Correction Analysis: Genetic correlation rg between EoE and other interested diseases/traits were estimated by LD score regression (LDSC) using GWAS summary statistics overlap with HapMap3 variants as recommended [14]. Pre-computed linkage disequilibrium scores for HapMap3 SNPs calculated based on European-ancestry individuals from the 1000 Genomes Project were used in the analysis, and SNP markers with an imputation INFO score<0.9 were excluded.
Gene Set Enrichment Analysis: Gene-based P-values were calculated with FUMA web server[15] using MAGMA[16] method based on the GWAS summary statistics. A total of 375 genes with a gene-based P-value less than 0.01 were considered as prioritized candidate genes for the downstream enrichment analysis. Gene set enrichment analysis was performed by GENE2FUNC pipeline implemented in FUMA web server.

The following examples are provided to illustrate certain embodiments of the invention. The examples are not intended to limit the invention in any way.

### EXAMPLE I

Here we describe the results of a well powered genome-wide association study (GWAS) of EoE including a total of 1,930 affected European subjects and 13,634 ancestry matched controls, identifying eleven (11) novel EoE associated loci and sex-specific susceptibility loci.

### Genome-Wide Significant Loci of EoE

GWAS was conducted in each of the four cohorts prior to meta-analysis. The previously reported loci at 2p23.1 (rs149864795, P=1.54×10⁻⁸, OR=1.87), 5q22.1 (rs1438673, P=7.82×10⁻¹⁰, OR=0.70) and 11q13.5 (rs61894547, P=4.22×10⁻⁸, OR=1.88) surpassed genome-wide significance in the CHOP-1 cohort. The 5p15.2 locus (rs116515942, P=4.22×10⁻⁹, OR=6.01) was significant in the CHOP-3 cohort, but missing in the CHOP-1 and CHOP-2 cohorts in part due to coverage issues. The 20q12 locus (rs62203849, P=1.53×10⁻⁸, OR=2.56) and the known locus at 2p23.1 (rs143457388, P=1.65×10⁻¹⁰, 0.65) were significant in the eMERGE cohort from CCHMC.

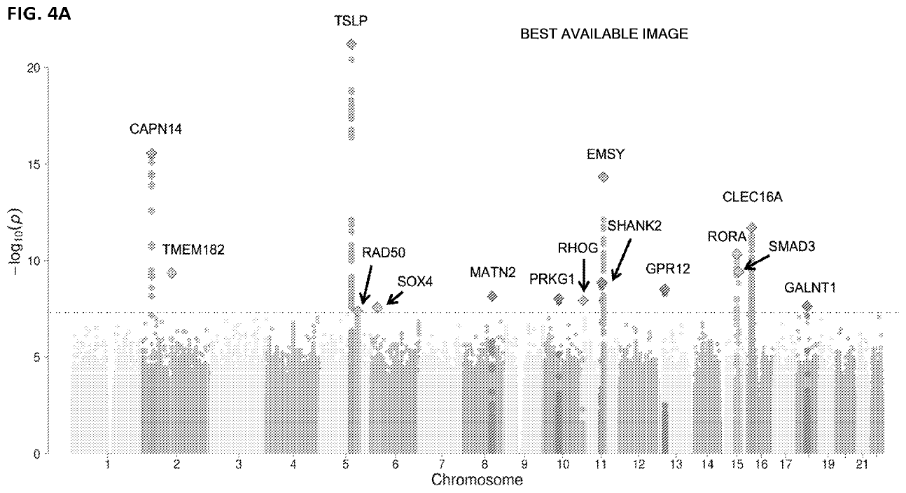

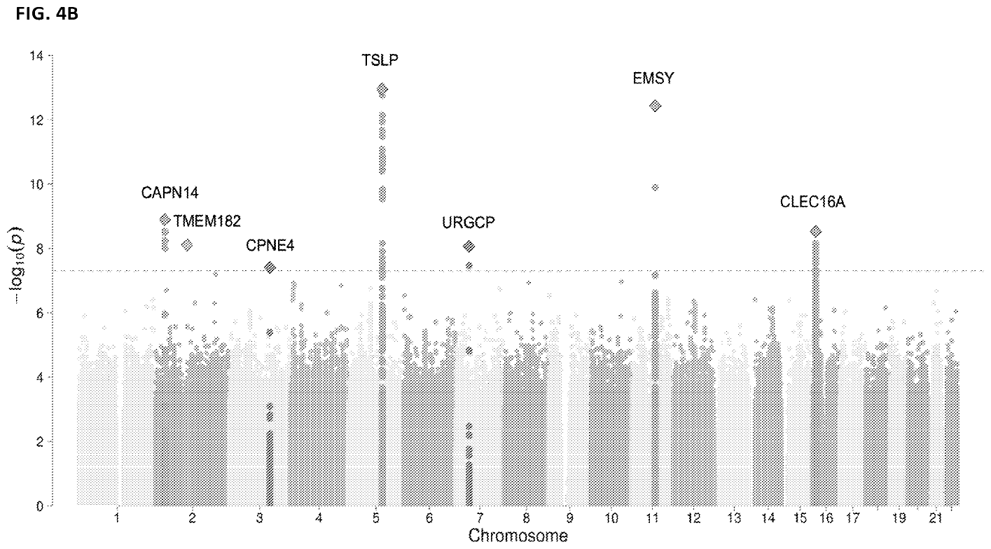

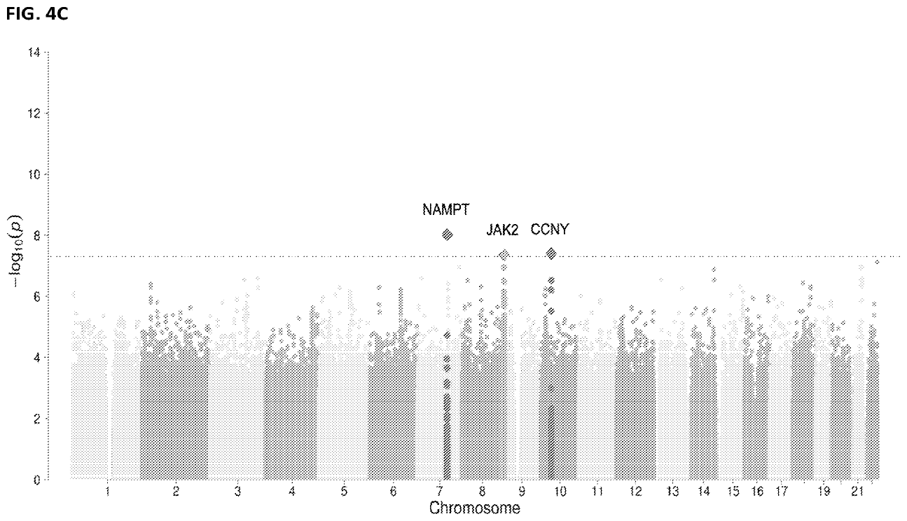

Meta-analysis of the results from the four cohorts detected 15 genome-wide significant loci associated with the risk of EoE (FIG. 4A-4C and Table 2).

TABLE 2
Results for genome-wide significant index variants at the 11
loci associated with EoE identified in the GWAS meta-analysis

| Locus | SNP | Gene | A1/A2 | OR | | P |
|---|---|---|---|---|---|---|
| 2p23.1 | rs143457388 | CAPN14** | A/T | 1.77 | [1.54, 2.03] | 2.69 × 10⁻¹⁶ |
| 2q12.1 | rs887992 | TMEM182 | C/A | 0.75 | [0.70 ,0.82] | 4.43 × 10⁻¹⁰ |
| 5q22.1 | rs1438673 | TSLP/WDR36** | C/T | 0.70 | [0.65, 0.76] | 6.12 × 10⁻²² |
| 5q31.1 | rs2106984 | RAD50 | A/T | 1.26 | [1.16, 1.37] | 4.11 × 10⁻⁸ |
| 6p22.3 | rs1620996 | SOX4 | T/C | 0.69 | [0.59, 0.78] | 2.7 × 10⁻⁸ |
| 8q22.1* | rs2513845 | MATN2 | T/C | 4.18 | [2.58, 6.79] | 6.98 × 10⁻⁹ |
| 10q21.1* | rs185811602 | PRKG1 | T/A | 6.37 | [3.38, 11.97] | 9.55 × 10⁻⁹ |
| 11p15.4 | rs147702004 | RHOG | T/A | 1.95 | [1.55, 2.45] | 1.15 × 10⁻⁸ |
| 11q13.4* | rs182139615 | SHANK2 | T/C | 6.62 | [3.59, 12.19] | 1.39 × 10⁻⁹ |
| 11q13.5 | rs61894547 | EMSY** | T/C | 1.79 | [1.55, 2.07] | 4.69 × 10⁻¹⁵ |
| 13q12.13* | rs146034499 | GPR12 | A/G | 5.92 | [3.29, 10.65] | 3.16 × 10⁻⁹ |
| 15q22.2 | rs2279293 | RORA | G/C | 0.69 | [0.61, 0.77] | 4.66 × 10⁻¹¹ |
| 15g23 | rs56062135 | SMAD3 | T/C | 1.29 | [1.19, 1.39] | 3.79 × 10⁻¹⁰ |
| 16p13.13 | rs35099084 | CLEC16A** | T/C | 0.72 | [0.65, 0.79] | 1.92 × 10⁻¹² |
| 18q12.2* | rs534845465 | GALNT1 | A/G | 5.78 | [3.12, 10.69] | 2.33 × 10⁻⁸ |

*Low-frequency loci
**Previously identified loci

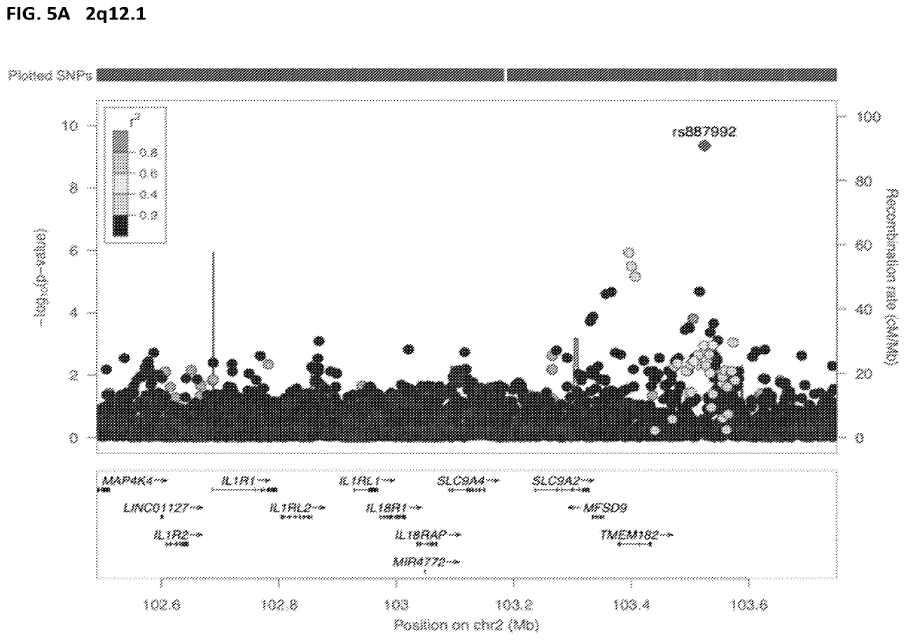

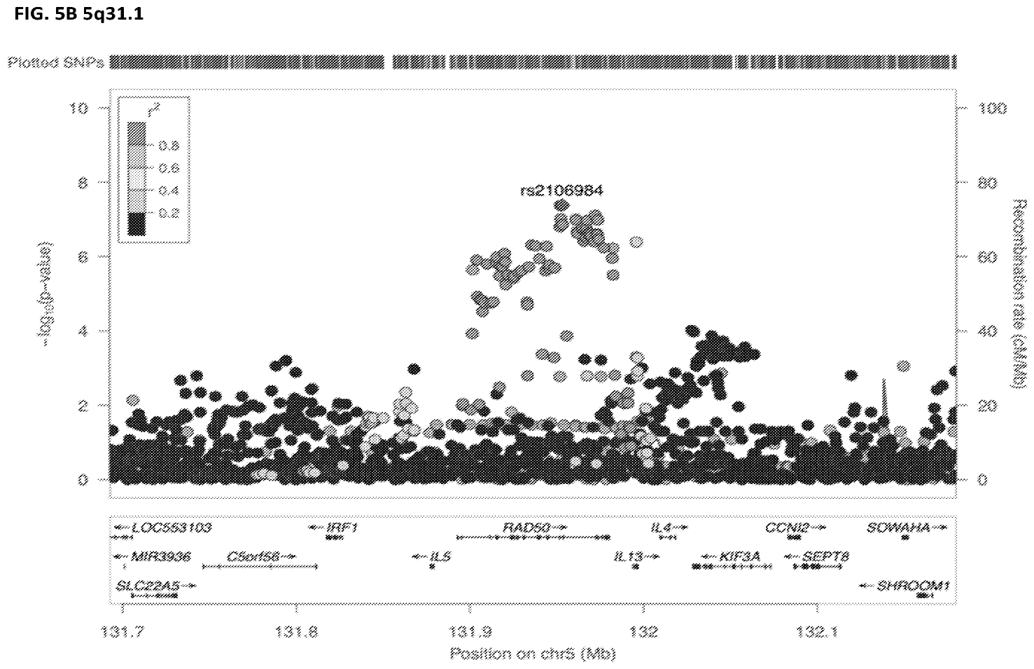

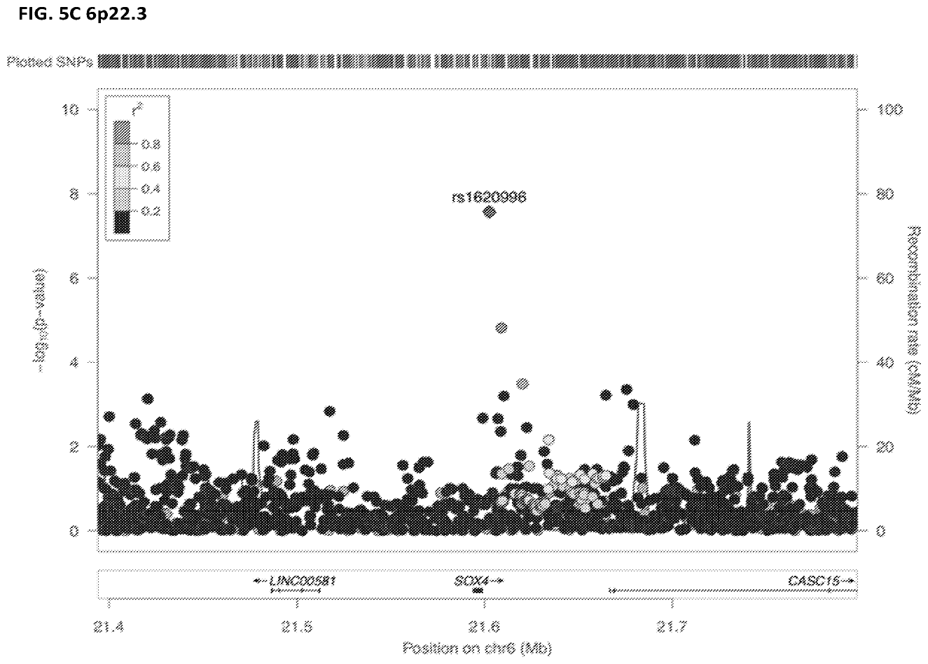

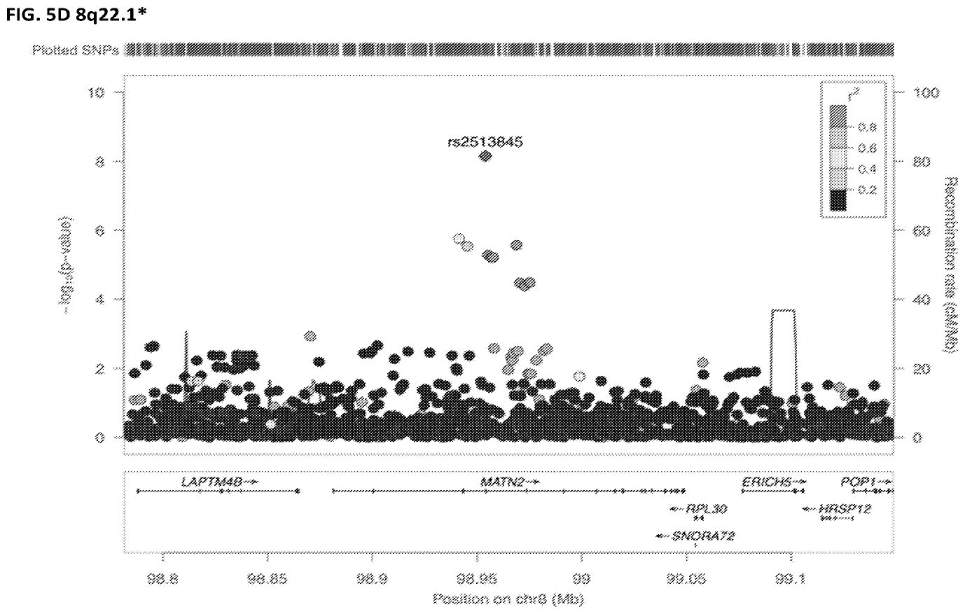

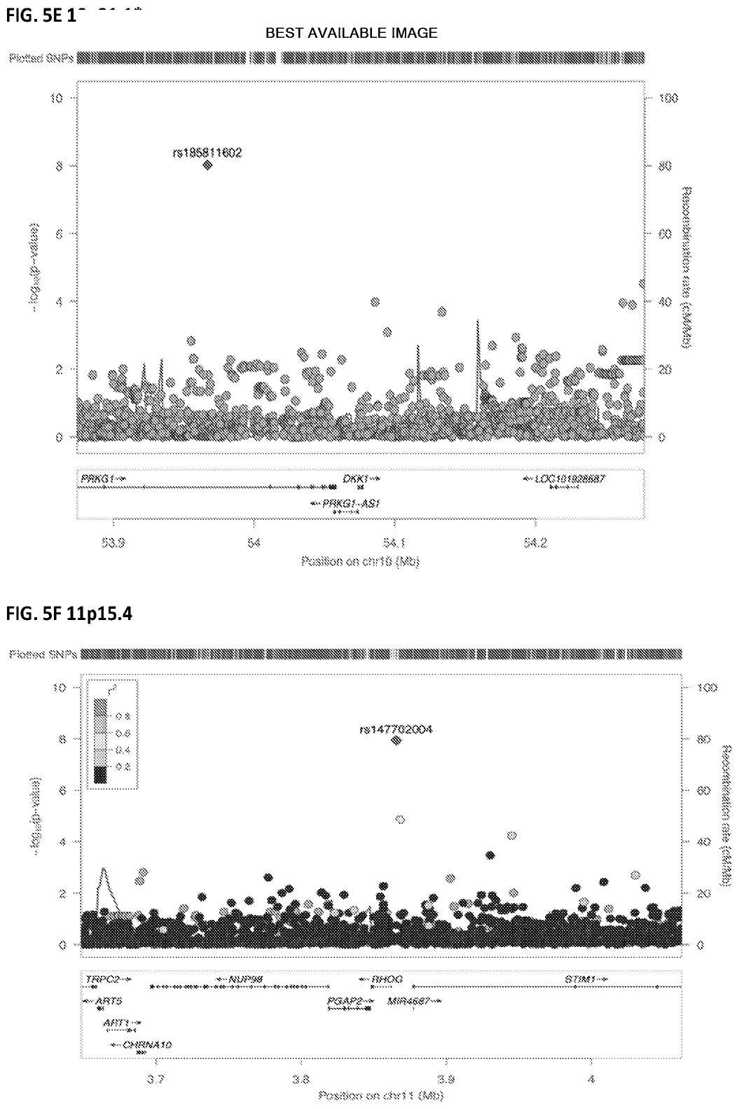

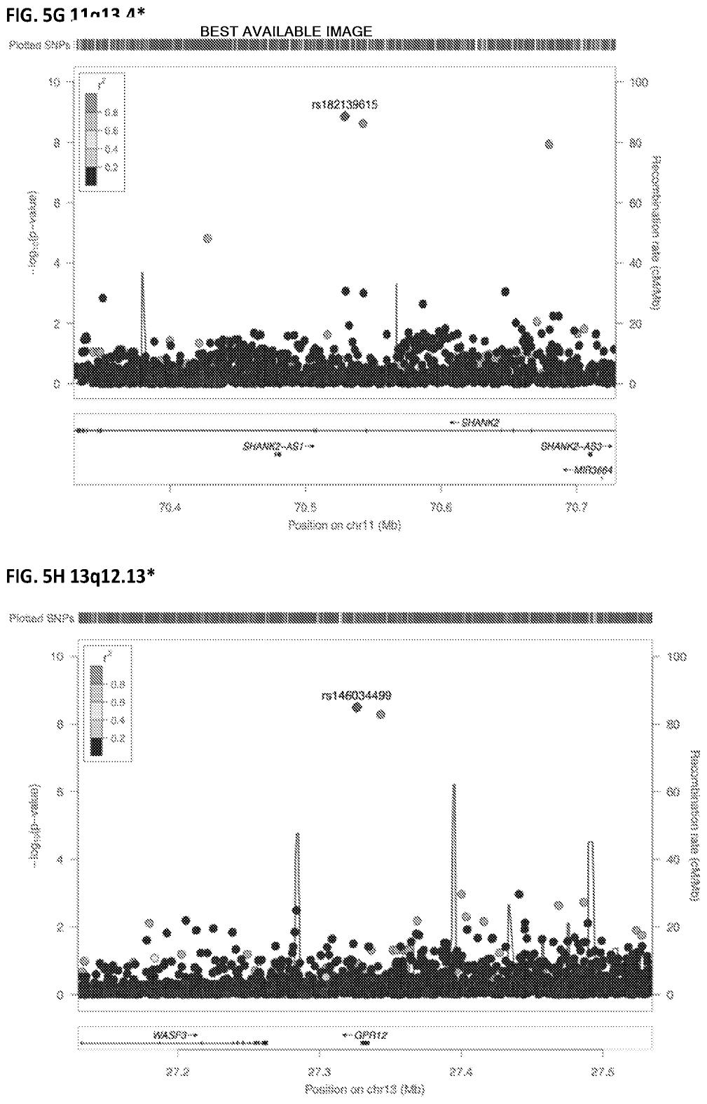

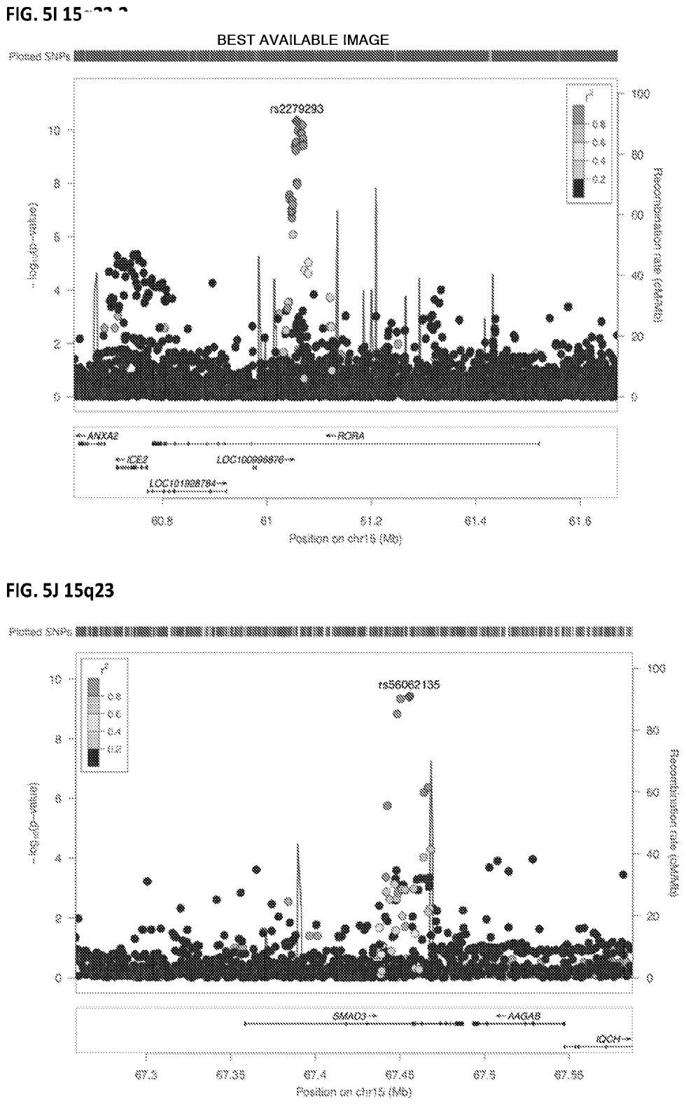

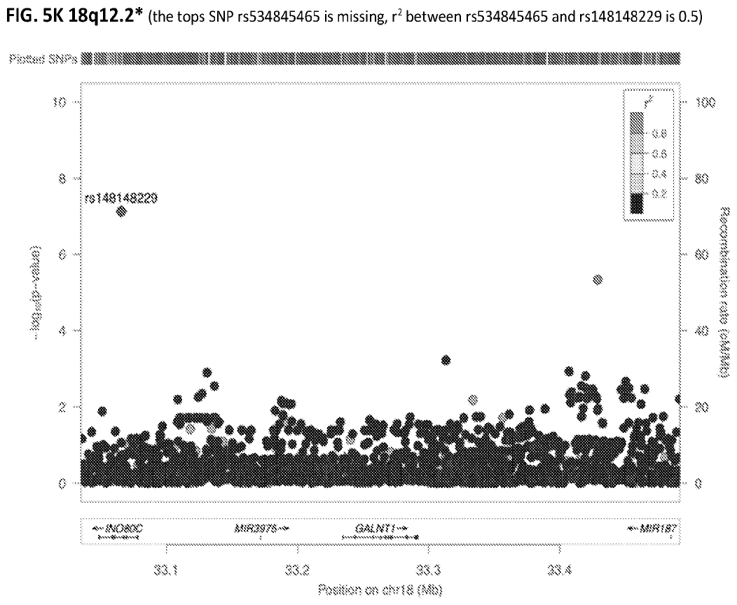

Besides the previously identified loci 2p23.1 (r5143457388, P=2.69×10⁻¹⁶, OR=1.77), 5q22.1 (rs1438673, P=6.12×10⁻²², OR=0.70) and 11q13.5 (rs61894547, P=4.69×10⁻¹⁵, OR=1.79), the locus at 16p13.13 (rs35099084, P=1.92×10⁻¹², 0.72), which was recently reported in a study using the Immunochip platform, replicated at a genome-wide significant level in our study (FIG. 4A-4C and Table 2). Among the 11 novel loci, 2q12.1 (rs887992, P=4.43×10⁻¹⁰, OR=0.75), 5q31.1 (rs2106984, P=4.11×10⁻⁸, OR=1.26), 6p22.3 (rs1620996, P=2.70×10⁻⁸, OR=0.69), 11p15.4 (rs147702004, P=1.15×10⁻⁸, OR=1.95), 15q22.2 (rs2279293, P=4.66×10^{-11,} OR=0.69), 15q23 (rs56062135, P=3.79×10^{-10, OR=}1.29) are all common variant loci, whereas 8822.1 (rs52513845, P=6.98×10⁻⁹, OR=4.18), 10q21.1 (rs185811602, P=9.55×10⁻⁹, OR=6.37), 11q13.4 (rs182139615, P=1.39×10⁻⁹, OR=6.62), 13q12.13 (rs146034499, P=3.16×10⁻⁹, OR=5.92), 18q12.2 (rs534845465, P=2.33×10⁻⁸, OR=5.78) are all low variant-frequency loci (FIG. 5A-5K and Table 3).

TABLE 3
Association Results of novel identified Risk Loci from the GWAS of 4 cohort

| Locus | | 2q12.1 | 5q31.1 | 6p22.3 | 8q22.1* | 10q21.1* | 11p15.4 |
|---|---|---|---|---|---|---|---|
| SNP | | rs887992 | rs2106984 | rs1620996 | r$2513845 | rs185811602 | rs147702004 |
| Gene | | TMEM182 | RAD50 | SOX4 | MATN2 | PRKG1 | RHOG |
| A1/A2 | | C/A | A/T | T/C | T/C | T/A | T/A |
| CHOP_HH | F1/F2 | 0.310/0.361 | 0.253/0.211 | 0.083/0.113 | 0.0098/0.0027 | 0.0046/0.0008 | 0.031/0.019 |
| | OR | 0.77 | 1.31 | 0.74 | 5.24 | 8.31 | 1.68 |
| | P | 3.25 × 10⁻⁵ | 6.63 × 10⁻⁵ | 2.53 × 10⁻³ | 1.96 × 10⁻⁶ | 3.19 × 10⁻⁴ | 3.48 × 10⁻³ |
| CHOP_OE | F1/F2 | NA | 0.253/0.212 | 0.085/0.121 | 0.0072/0.003 | 0.0042/0.001 | 0.022/0.017 |
| | OR | NA | 1.27 | 0.7 | 3.77 | 4.58 | 1.34 |
| | P | NA | 0.019 | 0.02 | 0.038 | 0.105 | 0.37 |
| CHOP_GSA | F1/F2 | NA | 0.231/0.205 | NA | NA | NA | NA |
| | OR | NA | 1.17 | NA | NA | NA | NA |
| | P | NA | 0.139739 | NA | NA | NA | NA |
| CCHMC | F1/F2 | 0.292/0.365 | 0.253/0.216 | 0.075/0.121 | 0.0085/0.0023 | 0.0094/0.0015 | 0.043/0.017 |
| | OR | 0.71 | 1.23 | 0.6 | 3.16 | 5.93 | 2.51 |
| | P | 2.17 × 10⁻⁶ | 6.45 × 10⁻³ | 1.95 × 10⁻⁵ | 6.03 × 10⁻³ | 2.52 × 10⁻⁵ | 1.64 × 10⁻⁷ |
| Combined | OR | 0.75 | 1.26 | 0.69 | 4.18 | 6.37 | 1.95 |
| | P | 4.43 × 10⁻¹⁰ | 4.11 × 10⁻⁸ | 2.7 × 10⁻⁸ | 6.98 × 10⁻⁹ | 9.55 × 10⁻⁹ | 1.15 × 10⁻⁸ |
| Locus | | 11q13.4* | 13q12.13* | 15q22.2 | 15q23 | 18q12.2* | 3q22.1*♂ |
| SNP | | rs182139615 | rs146034499 | rs2279293 | rs56062135 | rs534845465 | rs554318837 |
| Gene | | SHANK2 | GPR12 | RORA | SMAD3 | GALNT1 | CPNE4 |
| A1/A2 | | T/C | A/G | G/C | T/C | A/G | C/A |
| CHOP_HH | F1/F2 | 0.0059/0.0008 | 0.0048/0.0013 | 0.112/0.151 | 0.277/0.228 | 0.0059/0.0019 | 0.0201/0.0054 |
| | OR | 9.59 | 3.91 | 0.65 | 1.28 | 3.65 | 4.36 |
| | P | 1.59 × 10⁻⁵ | 5.48 × 10⁻³ | 2.72 × 10⁻³ | 1.11 × 10⁻⁴ | 3.20 × 10⁻³ | 1.23 × 10⁻⁶ |
| CHOP_OE | F1/F2 | 0.0085/0.001 | 0.0046/0.0013 | 0.104/0.153 | 0.256/0.225 | 0.0014/0.001 | NA |
| | OR | 9.6 | 11.53 | 0.7 | 1.18 | 1.9 | NA |
| | P | 6.75 × 10⁻³ | 3.2 × 10⁻³ | 7.78 × 10⁻⁵ | 0.108 | 0.614 | NA |
| CHOP_GSA | F1/F2 | 0.0036/0.0018 | 0.0085/0.001 | 0.101/0.146 | 0.293/0.221 | NA | 0.0102/0.0089 |
| | OR | 1.96 | 6.49 | 0.62 | 1.42 | NA | 1.22 |
| | P | 0.400144 | 0.013362 | 0.001682 | 0.000476 | NA | 0.72406 |
| CCHMC | F1/F2 | 0.0066/0.0011 | 0.0056/0.0013 | 0.104/0.136 | 0.281/0.233 | 0.0075/0.0012 | 0.0246/0.0094 |
| | OR | 6.67 | 6.94 | 0.72 | 1.28 | 11.71 | 2.53 |
| | P | 2.97 × 10⁻⁴ | 1.75 × 10⁻⁴ | 1.82 × 10⁻³ | 8.16 × 10⁻⁴ | 2.88 × 10⁻⁷ | 8.56 × 10⁻⁴ |
| Combined | OR | 6.62 | 5.92 | 0.69 | 1.29 | 5.78 | 2.88 |
| | P | 1.39 × 10⁻⁹ | 3.16 × 10⁻⁹ | 4.66 × 10⁻¹¹ | 3.79 × 10⁻¹⁰ | 2.33 × 10⁻⁸ | 3.86 × 10⁻⁸ |

| Locus | | 7p13*♂ | 7q22.3*♀ | 9p24.1♀ | 10p11.21*♀ |
|---|---|---|---|---|---|
| SNP | | rs188483654 | rs147307036 | rs62541556 | rs191051238 |
| Gene | | URGCP | NAMPT | JAK2 | CCNY |
| A1/A2 | | C/T | A/C | T/G | C/G |
| CHOP_HH | F1/F2 | 0.01/0.0027 | 0.0165/0.0026 | 0.347/0.251 | 0.0047/0.0006 |
| | OR | 5.17 | 8.05 | 1.58 | 6.94 |
| | P | 9.82 × 10⁻⁵ | 3.26 × 10⁻⁵ | 2.53 × 10⁻⁵ | 0.036 |
| CHOP_OE | F1/F2 | 0.008/0.001 | NA | NA | NA |
| | OR | 10.29 | NA | NA | NA |
| | P | 0.039 | NA | NA | NA |
| CHOP_GSA | F1/F2 | 0.0025/0.0013 | 0.0125/0.0024 | 0.356/0.25 | 0.0125/0.002 |
| | OR | 1.9 | 7.01 | 1.69 | 6.92 |
| | P | 0.578558 | 0.02514 | 0.002504 | 0.023638 |
| CCHMC | F1/F2 | 0.0116/0.0016 | 0.0276/0.0031 | 0.348/0.251 | 0.0243/0.0014 |
| | OR | 7.1 | 8.34 | 1.61 | 20.59 |
| | P | 1.18 × 10⁻⁴ | 2.52 × 10⁻⁶ | 2.22 × 10⁻⁴ | 9.53 × 10⁻⁹ |
| Combined | OR | 5.68 | 8.04 | 1.61 | 13.12 |
| | P | 8.68 × 10⁻⁹ | 9.59 × 10⁻⁹ | 4.4 × 10⁻⁸ | 3.95 × 10⁻⁸ |

A1/A2: minor allele/major allele;
F1/F2: minor allele frequency in cases/minor allele frequency in controls;
*low-frequency loci;
♂male-specific loci;
♀female-specific loci

### Sex-Stratified GWAS

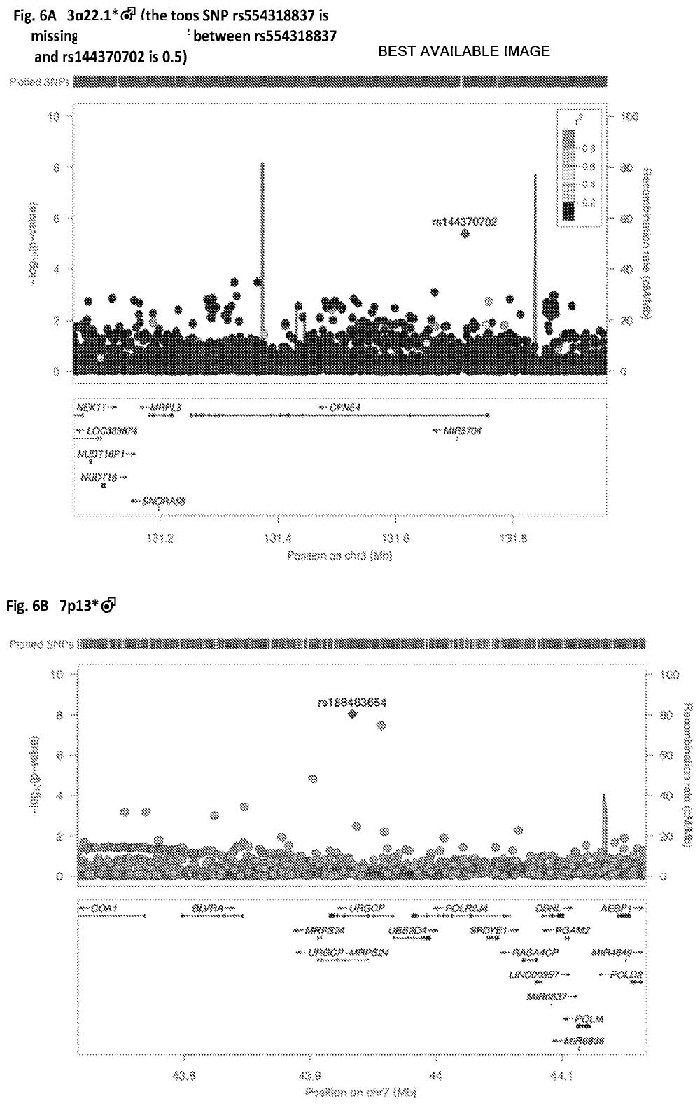

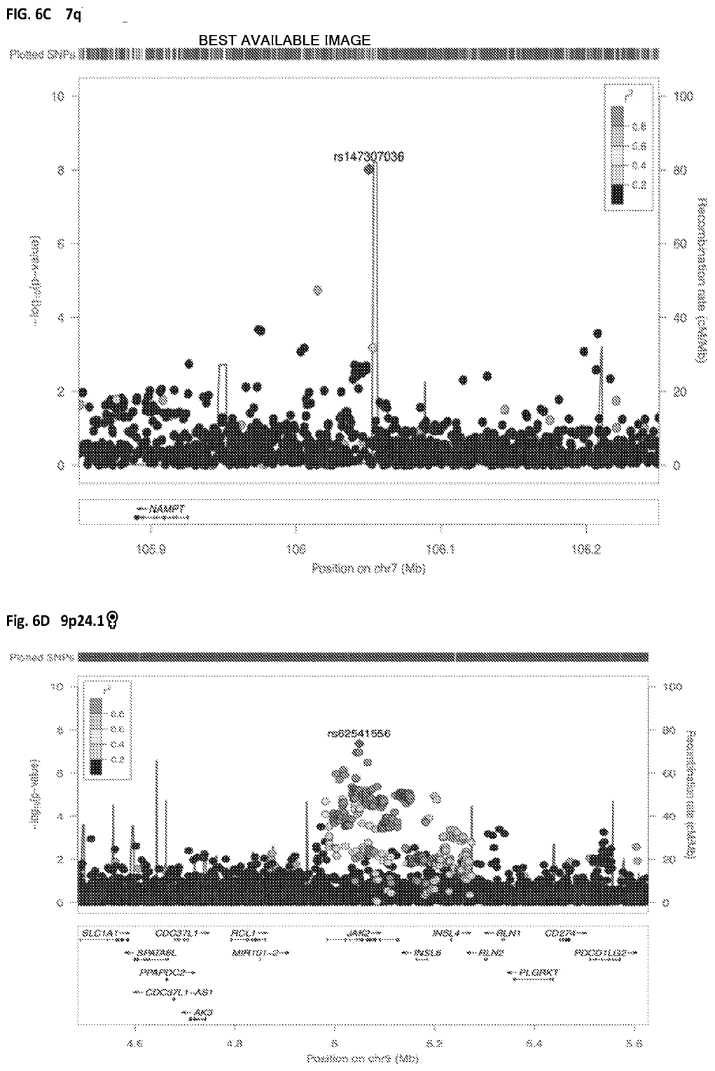

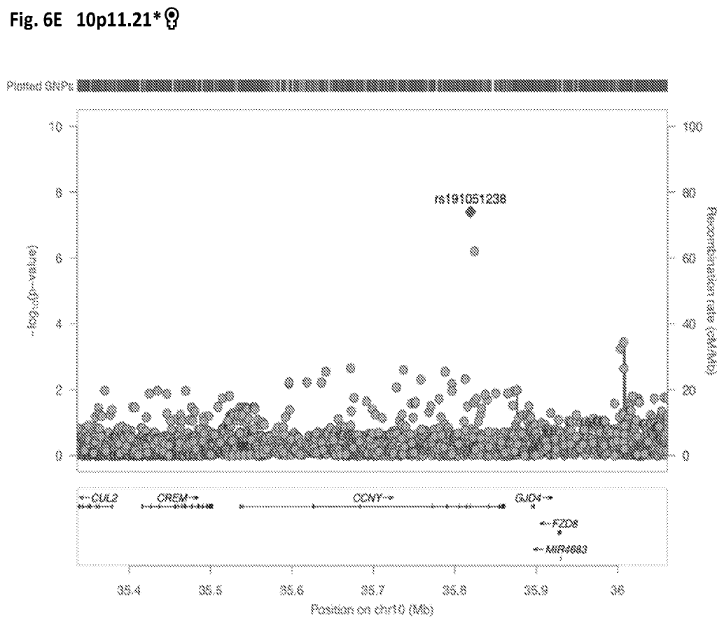

Considering observed sex differences in EoE, we performed meta-analyses of sex-stratified GWAS to identify sex-specific loci. Meta-analysis of males identified seven genome-wide significant loci, of which the low-frequency loci at 3q22.1 (rs554318837, P=3.86×10⁻⁸, OR=2.88) and 7p13 (rs188483654, P=8.68×10⁻⁹, OR=5.68) were male-specific (FIG. 4A-4C, Table 4, FIG. 6A-6E and Table 3). Meta-analysis of females detected two low variant-frequency loci at 7q22.3 (rs147307036, P=9.59×10⁻⁹, OR=8.04) and 10p11.21 (rs191051238, P=3.95×10⁻⁸, OR=13.12), as well as a common variant locus at 9p24.1 (rs62541556, P=4.4×10⁻⁸, OR=1.61), reaching genome-wide significance. All of them were female-specific (FIG. 4A-4C, Table 4, FIG. 6A-6E and Table 3).

TABLE 4
Results for the genome-wide significant index variants at the loci
associated with EoE identified in the sex-specific analysis.

| Gender | Locus | SNP | Gene | A1/A2 | Freq | ORₘₐₗₑ | Pₘₐₗₑ | OR_{female} | P_{female} | Pₕₑₜ |
|---|---|---|---|---|---|---|---|---|---|---|
| Male | 2p23.1 | rs143457388 | CAPN14** | A/T | 0.078/0.046 | 1.77 [1.54, | 2.69 × | 1.91 [1.52, | 1.55 × | 0.41 |
| (1288 | | | | | | 2.03] | 10⁻¹⁶ | 2.40] | 10⁻⁶ | |
| cases, | 2q12.1 | rs887992 | TMEM182 | C/A | 0.292/0.362 | 0.75 [0.70, | 4.43 × | 0.81 [0.69, | 0.03 | 0.25 |
| 6548 | | | | | | 0.82] | 10⁻¹⁰ | 0.95] | | |
| controls) | 3q22.1* | rs554318837 | CPNE4 | C/A | 0.0199/0.0076 | 2.88 [1.98, | 3.86 × | 0.71 [0.29, | 0.53 | 5.46 × |
| | | | | | | 4.21] | 10⁻⁸ | 1.77] | | 10⁻³ |
| | 5q22.1 | rs1438673 | TSLP/ | C/T | 0.419/0.506 | 1.43 [1.30, | 1.13 × | 1.41 [1.23, | 2.82 × | 0.02 |
| | | | WDR36** | | | 1.57] | 10⁻¹³ | 1.61] | 10⁻⁵ | |
| | 7p13* | rs188483654 | URGCP | C/T | 0.009/0.0019 | 5.68 [3.14, | 8.68 × | NA | NA | NA |
| | | | | | | 10.25] | 10⁻⁹ | | | |
| | 11q13.5 | rs61894547 | EMSY** | T/C | 0.076/0.04 | 1.92 [1.61, | 3.70 × | 1.65 [1.26, | 1.65 × | 0.35 |
| | | | | | | 2.28] | 10⁻¹³ | 2.15] | 10⁻³ | |
| | 16p13.13 | rs35099084 | CLEC16A** | T/C | 0.167/0.222 | 0.71 [0.64, | 3.01 × | 0.73 [0.62, | 1.51 × | 0.81 |
| | | | | | | 0.80] | 10⁻⁹ | 0.86] | 10⁻³ | |
| Female | 7q22.3* | rs147307036 | NAMPT | A/C | 0.0194/0.0028 | 0.72 [0.23, | 0.58 | 8.04 [4.36, | 9.59 × | 3.39 × |
| (542 | | | | | | 2.32] | | 14.85] | 10⁻⁹ | 10⁻⁴ |
| cases, | 9p24.1 | rs62541556 | JAK2 | T/G | 0.349/0.251 | 1.11 [1.00, | 0.04 | 1.61 [1.39, | 4.40 × | 2.49 × |
| 7146 | | | | | | 1.23] | | 1.87] | 10⁻⁸ | 10⁻⁴ |
| controls) | 10p11.21* | rs191051238 | CCNY | C/G | 0.0126/0.0012 | 1.65 [0.76, | 0.21 | 13.12 | 3.95 × | 4.96 × |
| | | | | | | 3.59] | | [5.95, | 10⁻⁸ | 10⁻⁵ |
| | | | | | | | | 28.93] | | |

*Low-Frequency Loci
**Previously identified loci
A1/A2: minor allele/major allele
Freq: minor allele frequency in cases/minor allele frequency in controls
Pₕₑₜ: gender_heterogenity_P-value(heterogeneity between genders)

### Genetic Correlation with Other Phenotypes

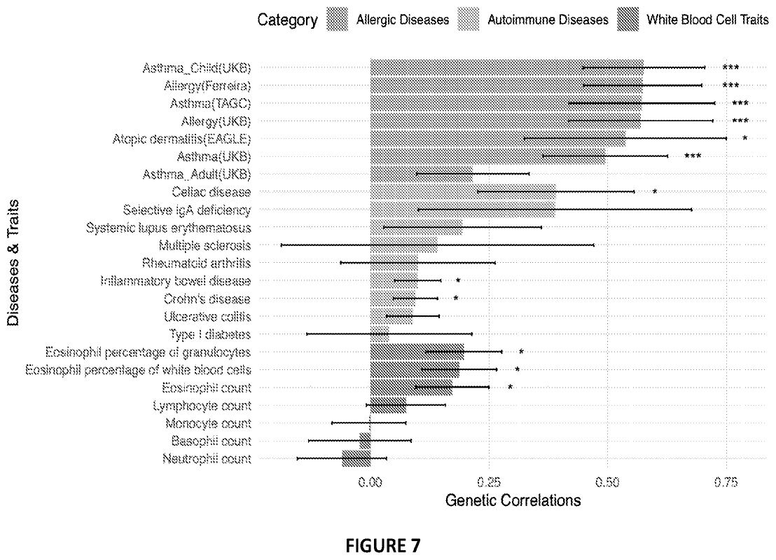

To investigate the genetic overlap between EoE and other phenotypes, we estimated the genetic correlations with EoE associated diseases and traits with summary statistics available from GWAS Catalog using LDSC (FIG. 7 and Table 5) [14, 17]. Significant correlations were observed between allergic diseases including allergy, asthma and atopic dermatitis and EoE (FIG. 7 and Table 5), and we also observed correlations between EoE and autoimmune diseases, including celiac disease and inflammatory bowel disease (FIG. 7 and Table 5). Moreover, blood cell traits, including eosinophil counts eosinophil percentage of granulocytes and eosinophil percentage of white blood cells were also significantly correlated with EoE.

TABLE 5
Genetic Correlations between EoE and Other Phenotypes

| Group | Disease | r_{g} | SE | P |
|---|---|---|---|---|
| Allergic | Asthma_Child (UKB) | 0.58 | 0.13 | 6.9 × 10⁻⁶ |
| Diseases | Allergy (Ferreira) | 0.57 | 0.12 | 4.08 × 10⁻⁶ |
| | Asthma (TAGC) | 0.57 | 0.15 | 2 × 10⁻⁴ |
| | Allergy (UKB) | 0.57 | 0.15 | 2 × 10⁻⁴ |
| | Atopic dermatitis (EAGLE) | 0.54 | 0.21 | 0.01 |
| | Asthma (UKB) | 0.49 | 0.13 | 2 × 10⁻⁴ |
| | Asthma_Adult (UKB) | 0.22 | 0.12 | 0.07 |
| Auto- | Celiac disease | 0.39 | 0.16 | 0.02 |
| immune | Selective IgA deficiency | 0.39 | 0.29 | 0.18 |
| Diseases | Systemic lupus | 0.19 | 0.17 | 0.24 |
| | erythematosus | | | |
| | Multiple sclerosis | 0.14 | 0.33 | 0.67 |
| | Rheumatoid arthritis | 0.10 | 0.16 | 0.54 |
| | Inflammatory bowel disease | 0.10 | 0.05 | 0.04 |
| | Crohn's disease | 0.09 | 0.05 | 0.04 |
| | Ulcerative colitis | 0.09 | 0.06 | 0.11 |
| | Type I diabetes | 0.04 | 0.17 | 0.82 |
| White | Eosinophil percentage of | 0.20 | 0.08 | 0.01 |
| Blood | granulocytes | | | |
| Cell | Eosinophil percentage of | 0.19 | 0.08 | 0.02 |
| Traits | white blood cells | | | |
| | Eosinophil counts | 0.17 | 0.08 | 0.03 |
| | Lymphocyte counts | 0.07 | 0.08 | 0.37 |
| | Monocyte count | −0.004 | 0.08 | 0.96 |
| | Basophil count | −0.023 | 0.11 | 0.83 |
| | Neutrophil count | −0.060 | 0.09 | 0.52 |

r_{g}: Genetic correlation

### Biological Annotation of Significant Loci

We examined whether the top SNPs identified at each of novel risk loci were associated with other phenotypes in previous GWAS. Notably, most of them were associated with allergic conditions and/or eosinophil count including three known risk loci for allergic diseases at 5q31.1 (rs2106984, RAD50), 15q22.2 (rs2279293, RORA) and 15q23 (rs56062135, SMAD3). The female-specific locus at 9p24.1 (rs62541556, JAK2) was also previously associated with inflammatory bowel disease, allergic diseases and eosinophil count at a genome-wide significance level. Interestingly, 13q12.13 (rs146034499, GPR12) was previously reported to be associated with both gastro-esophageal reflux and eosinophil count. The male-specific locus at 3q22.1 (rs554318837, CPNE4) was associated with both gastroesophageal reflux disease and allergy/anaphylactic reaction to food. The female-specific locus at 10p11.21 (rs191051238, CCNY) was associated with diseases of the esophagus, eosinophil count and age of onset of asthma diagnosis (Table 6).

TABLE 6
Diseases and Traits Associated with Novel Identified Loci in EoE GWAS

| Locus | SNP | Gene | Trait | P | | OR | Study |
|---|---|---|---|---|---|---|---|
| 2q12.1 | rs887992 | TMEM182 | Age hay fever, rhinitis | 8.6 × | 10⁻⁴ | 0.75 | UKB Neale v2 (2018) |
| | | | or eczema diagnosed | | | | |
| 5q31.1 | rs2106984 | RAD50 | Eosinophill count | 9.4 × | 10⁻¹³⁸ | 1.06 | UKB Neale v2 (2018) |
| | | | Eosinophill count | 1.7 × | 10⁻⁶⁸ | 1.08 | Astle WJ (2016) [1] |
| | | | Allergic disease | 7 × | 10⁻¹⁴ | 1.05 | Ferreira MA (2017) |
| | | | (asthma, hay fever or | | | | [2] |
| | | | eczema) | | | | |
| 6p22.3 | rs1620996 | SOX4 | Extrinsic allergic | 9.5 × | 10⁻³ | 0.51 | UKB SAIGE (2018) |
| | | | alveolitis | | | | [3] |
| 8q22.1* | rs2513845 | MATN2 | Age hay fever, rhinitis | 4 × | 10⁻³ | 0.028 | UKB Neale v2 (2018) |
| | | | or eczema diagnosed | | | | |
| 10q21.1* | rs185811602 | PRKG1 | Age asthma diagnosed | 0.046 | | 8.6 × | UKB Neale v2 (2018) |
| | | | by doctor | | | 10⁻⁴ | |
| 11p15.4 | rs147702004 | RHOG | Age hay fever, rhinitis | 0.042 | | 3.97 | UKB Neale v2 (2018) |
| | | | or eczema diagnosed | | | | |
| 11q13.4* | rs182139615 | SHANK2 | NA | NA | | | NA |
| 13q12.13* | rs146034499 | GPR12 | Gastro-oesophageal | 1.5 × | 10⁻³ | 1.57 | UKB Neale v2 (2018) |
| | | | reflux (gord)/gastric | | | | |
| | | | reflux | | | | |
| | | | Eosinophil counts | 7.6 × | 10⁻³ | 1.11 | Astle WJ (2016) [1] |
| 15q22.2 | rs2279293 | RORA | Age hay fever, rhinitis | 8.5 × | 10⁻¹⁰ | 2.1 | UKB Neale v2 (2018) |
| | | | or eczema diagnosed | | | | |
| | | | Eosinophill count | 1.6 × | 10⁻⁹ | 0.98 | UKB Neale v2 (2018) |
| | | | Eosinophil counts | 1.7 × | 10⁻⁷ | 0.97 | Astle WJ (2016) [1] |
| | | | Allergic disease | 1.3 × | 10⁻⁸ | 0.95 | Ferreira MA (2017) |
| | | | (asthma, hay fever or | | | | [2] |
| | | | eczema) | | | | |
| | | | Doctor diagnosed | 2.2 × | 10⁻⁸ | 0.89 | UKB Neale v2 (2018) |
| | | | asthma | | | | |
| | | | Doctor diagnosed | 9.4 × | 10⁻⁸ | 0.92 | UKB Neale v2 (2018) |
| | | | hayfever or allergic | | | | |
| | | | rhinitis | | | | |
| | | | Inflammatory bowel | 2.7 × | 10⁻⁶ | 0.92 | de Lange KM (2017) |
| | | | disease | | | | [4] |
| 15q23 | rs56062135 | SMAD3 | Allergic disease | 3.4 × | 10⁻²⁷ | 1.08 | Ferreira MA (2017) |
| | | | (asthma, hay fever or | | | | [2] |
| | | | eczema) | | | | |
| | | | Eosinophill count | 1.1 × | 10⁻²² | 1.02 | UKB Neale v2 (2018) |
| | | | Eosinophil counts | 8 × | 10⁻¹⁰ | 1.03 | Astle WJ (2016) [1] |
| | | | Inflammatory bowel | 1.4 × | 10⁻²¹ | 1.15 | de Lange KM (2017) |
| | | | disease | | | | [4] |
| | | | Crohn's disease | 8.9 × | 10⁻¹⁹ | 1.18 | de Lange KM (2017) |
| | | | | | | | [4] |
| | | | Ulcerative colitis | 4.7 × | 10⁻⁹ | 1.11 | de Lange KM (2017) |
| | | | | | | | [4] |
| | | | Asthma | 3.4 × | 10⁻²¹ | 1.11 | UKB SAIGE (2018) |
| | | | | | | | [3] |
| 18q12.2* | rs534845465 | GALNT1 | NA | NA | | | NA |
| 3q22.1*♂ | rs554318837 | CPNE4 | Gastroesophageal | 2.5 × | 10⁻³ | 1.22 | UKB SAIGE (2018) |
| | | | reflux disease | | | | |
| | | | Allergy or anaphylactic | 9.2 × | 10⁻³ | 1.63 | UKB Neale v2 (2018) |
| | | | reaction to food (self- | | | | |
| | | | reported) | | | | |
| 7p13*♂ | rs188483654 | URGCP | Asthma (self-reported) | 6.1 × | 10⁻⁴ | 1.32 | UKB Neale v2 (2018) |
| | | | Inflammatory bowel | 0.038 | | 0.61 | UKB SAIGE (2018) |
| | | | disease and other | | | | [3] |
| | | | gastroenteritis and | | | | |
| | | | colitis | | | | |
| 7q22.3*♀ | rs147307036 | NAMPT | Recent medication for | 0.025 | | 1.94 | UKB Neale v2 (2018) |
| | | | asthma | | | | |
| 9p24.1♀ | rs62541556 | JAK2 | Inflammatory bowel | 2.1 × | 10⁻³¹ | 1.14 | Liu JZ (2015) [5] |
| | | | disease | | | | |
| | | | Inflammatory bowel | 6.1 × | 10⁻²⁶ | 1.16 | de Lange KM (2017) |
| | | | disease | | | | [4] |
| | | | Ulcerative colitis | 7.9 × | 10⁻²¹ | 1.14 | Liu JZ (2015) [5] |
| | | | Ulcerative colitis | 1.1 × | 10⁻¹⁶ | 1.16 | de Lange KM (2017) |
| | | | | | | | [4] |
| | | | Crohn's disease | 4.7 × | 10⁻²⁰ | 1.13 | Liu JZ (2015) [5] |
| | | | Crohn's disease | 1.3 × | 10⁻¹⁶ | 1.16 | de Lange KM (2017) |
| | | | | | | | [4] |
| | | | Eosinophill count | 6.2 × | 10⁻²⁶ | 1.02 | UKB Neale v2 (2018) |
| | | | Eosinophil counts | 5.2 × | 10⁻¹⁵ | 1.03 | Astle WJ (2016) [1] |
| | | | Allergic disease | 1.2 × | 10⁻⁶ | 1.03 | Ferreira MA (2017) |
| | | | (asthma, hay fever or | | | | [2] |
| | | | eczema) | | | | |
| 10p11.21*♀ | rs191051238 | CCNY | Diseases of esophagus | 0.026 | | 0.86 | UKB SAIGE (2018) |
| | | | | | | | [3] |
| | | | Eosinophil counts | 0.038 | | 1.08 | Astle WJ (2016) [1] |
| | | | Age asthma diagnosed | 4.5 × | 10⁻³ | 2.7 × | UKB Neale v2 (2018) |
| | | | by doctor | | | 10⁻³ | |

*Low-Frequency Loci

We used the HaploReg web server to investigate the potential functional consequences of the top SNP and SNPs in the same LD block from each novel locus [18]. HaploReg annotations indicated the tested SNPs were highly enriched in regulatory regions of the genome. Among the 16 assessed loci, 12 loci were associated with enhancer activities, six loci were associated with promoter histone markers, nine loci were located in DNase I hypersensitive regions, and 14 loci were located in regulatory motifs. Interestingly, the most significant SNP, rs2513845, from the low-frequency locus 8q22.1 was a synonymous mutation of gene MATN2, and also was located in the conserved region.

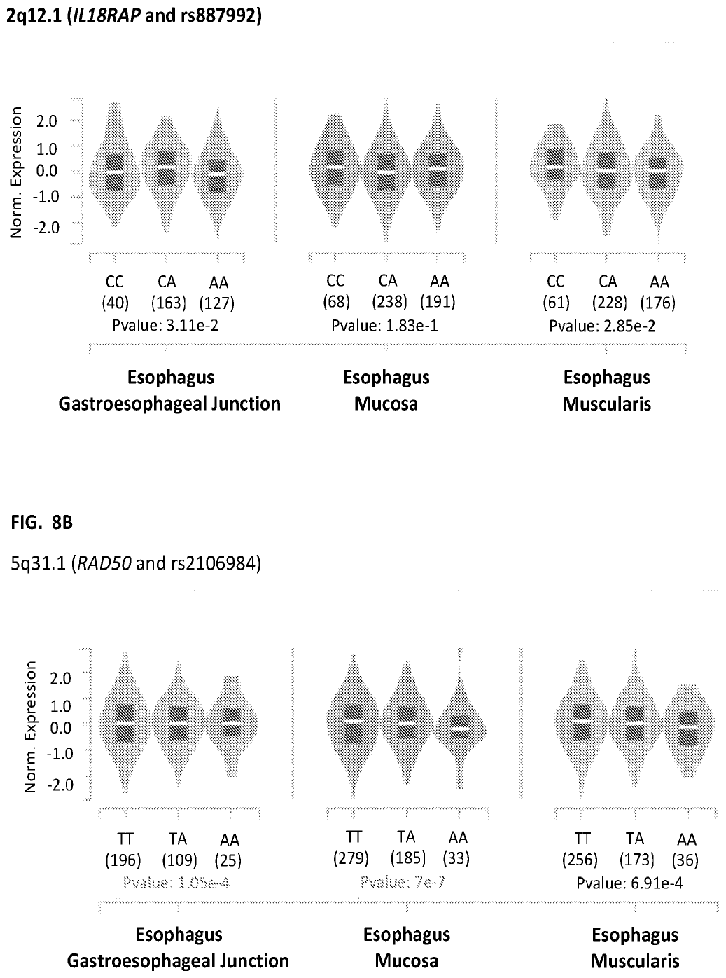

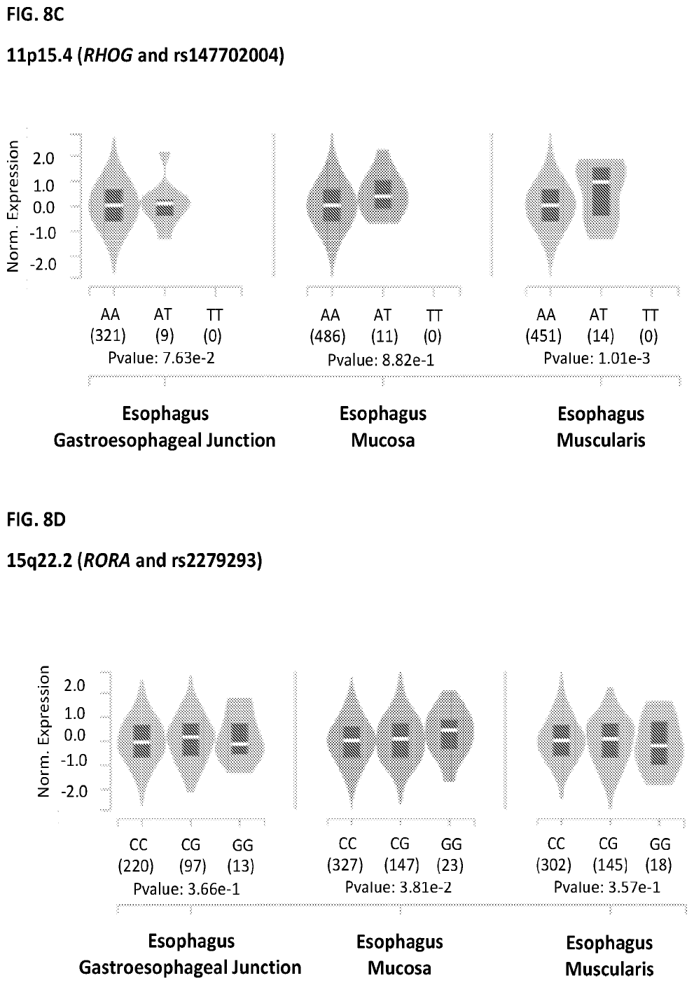

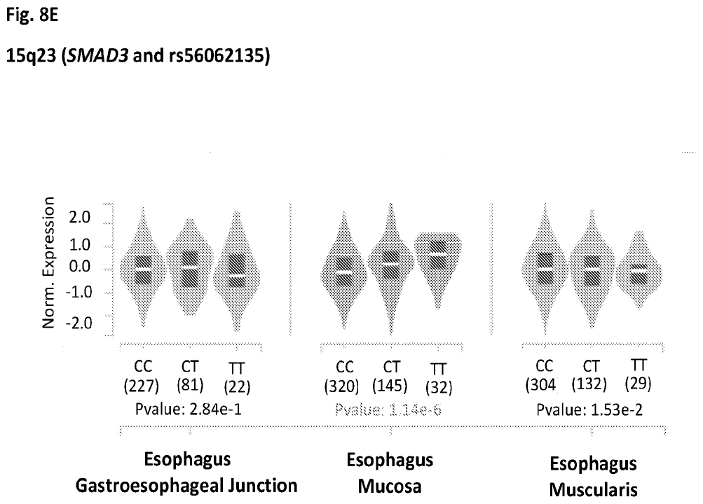

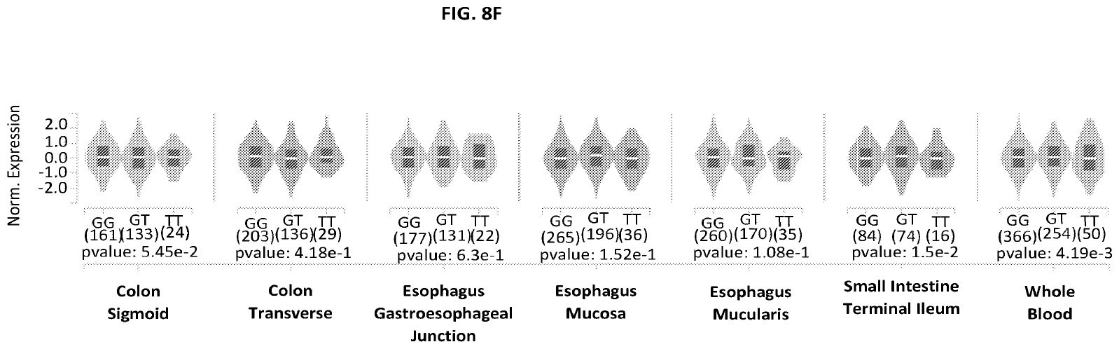

We next examined if genotypes of the top SNP identified at the genome-wide significant loci were correlated with the mRNA expression levels of its nearby genes in tissues of the esophagus using GTEx Portal. The top SNP, rs2106984, at 5q31.1 is an eQTL for RAD50 at the esophagus gastroesophageal junction and in the esophagus mucosa (FIG. 8a-8F). The top SNP, rs56062135, at 15q23 is an eQTL for SMAD3 in the esophageal mucosa (FIG. 8A-8F). Although not meeting the 5% false discovery rate, the rs2279293 genotypes at 15q22.2 were correlated with RORA expression levels in the esophageal mucosa, the rs147702004 genotypes at 11p15.4 were correlated with RHOG expression levels in the esophageal mucosa, and the rs887992 genotypes at 2q12.1 were correlated with IL18RAP expression levels in the esophagus, at the gastroesophageal junction and in the muscularis layer of the esophagus (FIG. 8A-8F). In addition, top SNP at the female-specific locus 9p24.1 is an eQTL for the pseudogene IGHEP2 in multiple tissues including the esophagus and at the gastroesophageal junction (data not shown). Modestassociation was detected between the rs62541556 genotypes and expression levels of JAK2 at 9p24.1 in whole blood and small intestine terminal ileum (FIG. 8A-8F).

### Analysis of Gene Sets

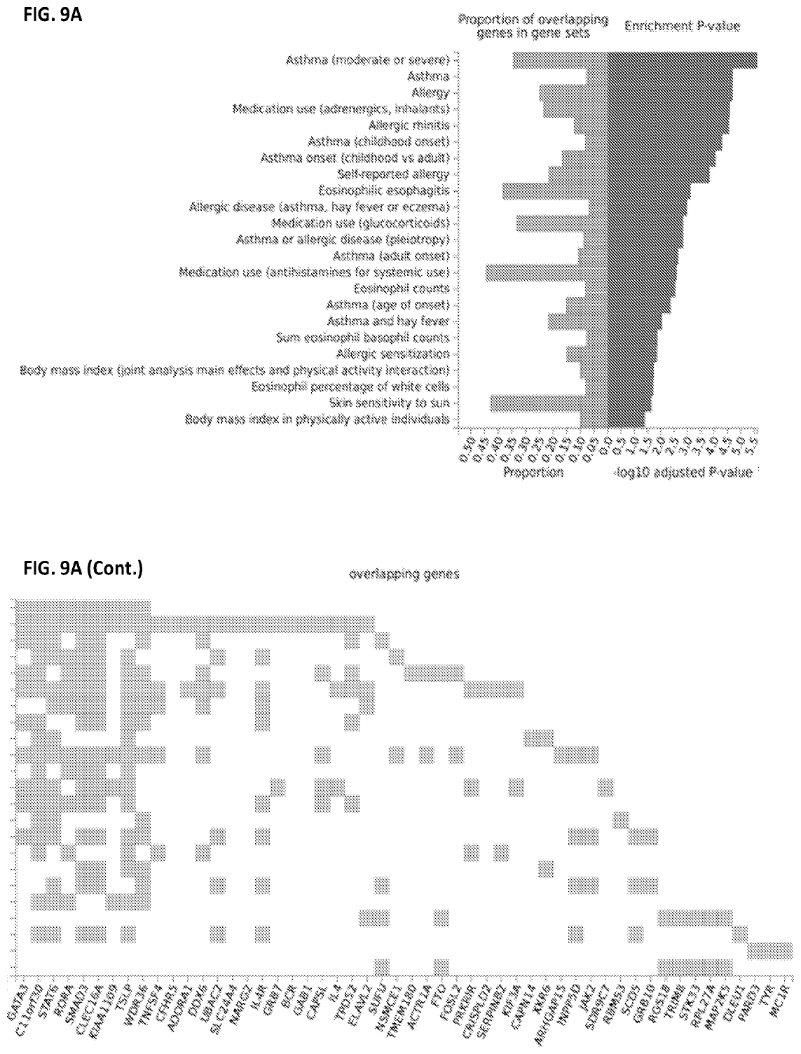

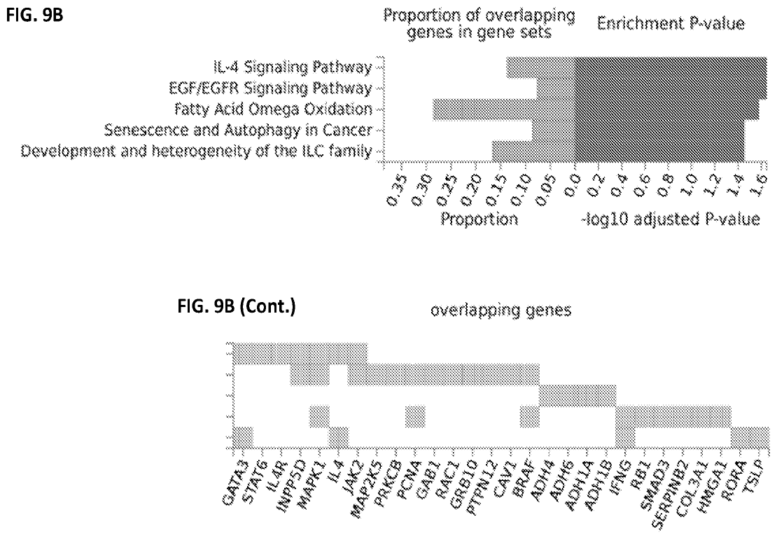

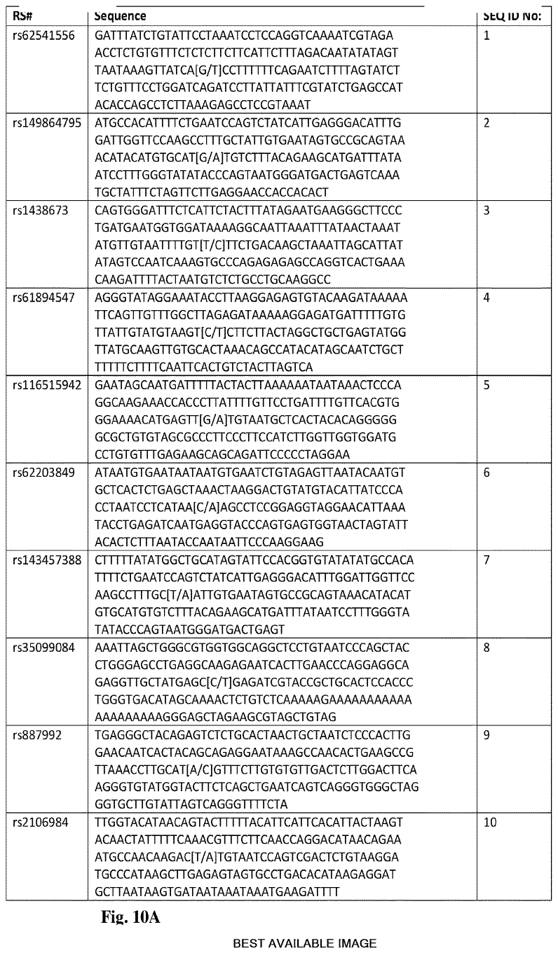

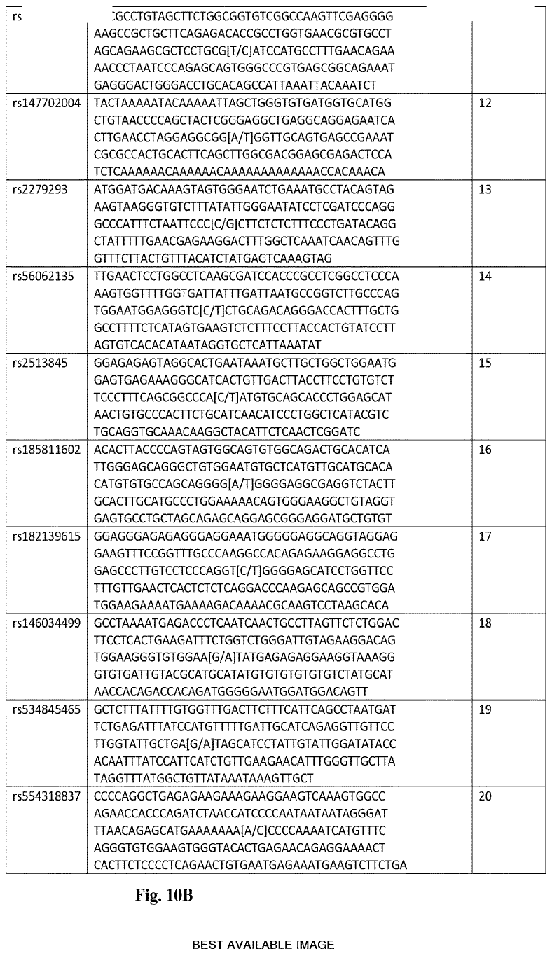

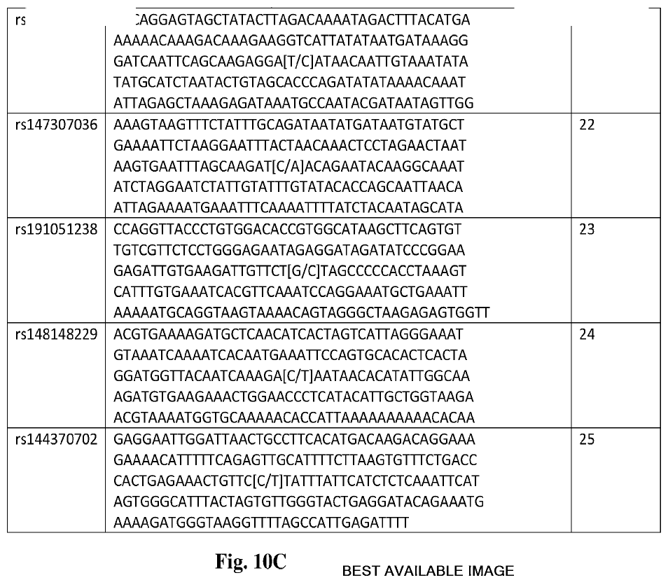

We conducted a gene-based association analysis of our EoE meta-analysis using the FUMA web server[15] and MAGMA[16], selecting 375 candidate genes with a gene-based P value less than 0.01 for the gene set enrichment analysis. Analysis of the GWAS Catalog reported genes showed a high consistency with results from our genetic correlation analysis that genes involved in allergy associated diseases/traits and eosinophil associated traits were significantly overrepresented (FIG. 9A-9B). Enrichment results based on WikiPathways further indicated that candidate genes were significantly enriched in gene pathways involving IL-4 signaling, and in the development and heterogeneity of the innate lymphoid cell family of genes (FIG. 9A-9B).

### DISCUSSION

Here, we report the results of the largest EoE GWAS to date with a sample size of 2048 cases and 12,429 controls. Our analysis identified 15 risk loci for EoE of which 11 are novel. The replication of known loci, including 2p23.1 (CAPN14), 5q22.1 (TSLP) and 11q13.5 (EMSY/LRRC32), was expected given the amount of overlap between samples. However, the 16p13.13 (CLEC16A) locus was only detected previously from independent samples genotyped using the Immunochip platform. Here, our replication of the 16p13.13 locus (CLEC16A) using the universal Illumina SNP arrays supports the robustness of our analysis.

Similar to the previously reported loci at 5q22.1 (TSLP), 11q13.5 (EMSY/LRRC32) and 16p13.13 (CLEC16A), the newly detected loci at 5q31.1 (RAD50), 15q22.2 (RORA) and 15q23 (SMAD3) have been previously associated with allergic diseases, albeit with much lower odds ratios (Table 6), indicating a potentially more critical role of these loci in the development of EoE than other allergic disorders. eQTL analysis also indicated a significant association between the top SNP at 15q23 and SMAD3 expression levels in the esophageal mucosa, with a more modest association between the top SNP at 15q22.2 and RORA expression levels in the esophageal mucosa (FIG. 8A-8F). Both SMAD3 and RORA have been shown to be involved in immune associated pathways. For the 5q31.1 locus, the top SNP is an eQTL for RAD50 in the esophagus, the gastroesophageal junction and the esophageal mucosa, suggesting a potential role of RAD50 in EoE pathogenesis (FIG. 8A-8F). Regarding other nearby genes, including IL3, IL4, and IL5 at the 5q31.1 locus, gene expression was lacking for these genes in the esophagus based on the GTEx Portal. Indeed, IL3, IL4 and IL5 are primarily involved with biological processes involving the immune system and inflammatory responses, and linked to multiple allergic diseases.

At the 11p15.4 locus, a modest association was found between the top SNP and RHOG expression levels in the esophageal mucosa (FIG. 8A-8F). RHOG encodes RhoG, a member of the Rac subfamily of Rho GTPases highly expressed in lymphocytes. Cumulative evidence indicates RhoG is a key player in B/T cell phagocytosis. At the 2q12.1 locus, we observed a modest association between the top SNP and IL18RAP expression levels in the esophagus, at the gastroesophageal junction and in the muscularis layer of the esophagus (FIG. 8A-8F), but not to other nearby genes such as TMEM182 and MFSD9. This observation prioritizes the IL18RAP gene among its neighbors as a candidate gene for EoE. Moreover, IL18RAP has been associated with celiac disease and IBD. It also displays epigenetic dysregulation in childhood food allergy. The lead SNP at the 6p22.3 locus is adjacent to the SOX4 gene, which participates in multiple immune response pathways.

Besides the aforementioned six common loci, we identified five low-frequency loci by taking advantage of the TOPMed imputation reference panel, which substantially improved the imputation quality for low-frequency variants. Remarkably, the lead SNP, rs2513845 at the 8q22.1 locus is a synonymous variant of within the MATN2 gene. Although synonymous mutations do not directly cause amino acid changes in the encoded product, they can have an effect on mRNA stability and translation kinetics leading to significant biological consequences. MATN2 has been reported to induce inflammatory responses, and mediate M2 polarization and regulatory T-cells differentiation in allergic rhinitis. In addition, the lead SNP, rs185811602, at the 10q21.1 locus resides in the intronic region of PRKG1. PRKG1 encodes the cGMP-dependent protein kinase 1, which has been implicated in multiple inflammatory processes.

Our study revealed two low-frequency loci in males only. The top SNP at the 3q22.1 locus is located in the intronic region of CPNE4, and is previously associated with both gastroesophageal reflux disease and allergy/anaphylactic reaction to food (Table 6). The top SNP at the 7p13 locus is located in the intronic region of URGCP, and previously associated with self-reported asthma and Inflammatory bowel disease and other gastroenteritis and colitis (Table 6).

We also identified a common locus and two low-frequency loci that are specific in females. Interestingly, the common locus at 9p24.1 has been associated with IBD and allergic diseases (Table 6). Multiple lines of evidence indicated JAK2 played a crucial role in the pathogenesis through the JAK-STAT signaling pathway. Although the genotypes of the top SNP, rs62541556, were not associated with expression levels of JAK2 in the esophagus according to the GTEx portal, we cannot rule out the potential role of JAK2 in EoE. It also worth mentioning that the top SNP at the 10p11.21 low frequency locus is located in the intronic region of CCNY, and also associated with diseases of the esophagus, eosinophil count and age of asthma diagnosis (Table 6).

Our genetic correlation analysis uncovered strong and significant genetic overlap between EoE and various atopic conditions (FIG. 7 and Table 5). This observation suggested a considerable genetic component in the epidemiological correlation between EoE and other atopic conditions. Interestingly, and consistent with previous reports suggesting that genetic etiologies are partly distinct between childhood-onset and adult-onset asthma, we found the genetic correlation between EoE and childhood-onset asthma (r_{g}=0.58, P=6.9×10⁻⁶) is much stronger than adult-onset asthma (r_{g}=0.22, P=0.07). This observation can be explained by the predominant proportion of pediatric patients in our GWAS cohorts. In addition to allergic diseases, patients with EoE also exhibited increased risks for multiple autoimmune conditions. Likewise, we found positive genetic correlations between EoE and all the assessed autoimmune diseases (FIG. 7 and Table 5). The significant correlations were detected in autoimmune diseases affecting digestive system including Celiac disease (r_{g}=0.39, P=0.02) and IBD (r_{g}=0.1, P=0.04). As EoE is characterized by eosinophilic infiltration of the esophagus, we further estimated the genetic correlation between EoE and serum eosinophil levels and identified significant positive correlation (FIG. 7 and Table 5). However, no significant genetic correlation was observed between EoE and the levels of other types of white blood cells, suggesting there may be a specific underlying genetic link between EoE and eosinophil levels.

Along with genetic correlation, enrichment analysis of candidate genes from GWAS also pointed to shared genetic mechanisms among EoE, eosinophil levels and other allergic diseases. Enrichment analysis further suggested a potential role of the IL-4 signaling pathway, and development and heterogeneity of the innate lymphoid cell family in the pathogenesis of EoE. Indeed, both pathways have been linked to EoE or other atopic conditions.

Our study provides a set of gene targets that are associated with EoE, including the identification of 11 novel risk loci and six sex-specific risk loci. Our results also highlighted a substantial genetic overlap between EoE and phenotypes of atopic conditions, autoimmune diseases affecting the digestive system, and eosinophil levels, thereby providing deeper understanding of the genetic architecture and etiology of this devastating disease that has been growing rapidly in incidence and prevalence in recent years.

### EXAMPLE II

The information herein above can be applied clinically to patients for diagnosing an increased susceptibility for developing EoE, and the related autoimmune disorders described herein and therapeutic intervention. A preferred embodiment of the invention comprises clinical application of the information described herein to a patient. Diagnostic compositions, including microarrays, and methods can be designed to identify the genetic alterations described herein in nucleic acids from a patient to assess susceptibility for developing EoE. This can occur after a patient arrives in the clinic; the patient has blood drawn, and using the diagnostic methods described herein, a clinician can detect a SNP in the 11 risk loci described herein and listed in the claims, e.g., one or more of the genes shown in FIGS. 4-8. Patients may be of any age, while the typical age range for a pediatric patient to be screened is between 9 and 12 years of age. The information obtained from the patient sample, which can optionally be amplified prior to assessment, will be used to diagnose a patient with an increased or decreased susceptibility for developing EoE and other disorders. Kits for performing the diagnostic method of the invention are also provided herein. Such kits comprise a microarray comprising at least one of or all of the SNPs provided herein in and the necessary reagents for assessing the patient samples as described above.

In another approach, the patient may have had previous genotyping performed and this genetic information is stored in a computer data file. Accordingly, the methods of the invention may be performed in silico wherein the SNP containing reference sequences are compared with patient sequences electronically stored to assess the same for the presence or absence of the SNPS disclosed herein thereby diagnosing an increased or decreased risk for developing EoE.

The identity of EoE-involved genes and the patient results will indicate which variants are present, and will identify those that possess an altered risk for developing EoE. The information provided herein allows for therapeutic intervention at earlier times in disease progression that previously possible. Also as described herein above, the genes described herein and depicted in the Figures provide novel targets for the development of new therapeutic agents efficacious for the treatment of EoE and other immune disorders.

### REFERENCES

1 Dellon E S, Jensen E, Martin C F, Shaheen N J, Kappelman M D. Prevalence of eosinophilic esophagitis in the United States. Clin Gastroenterol Hepatol 2014; 12:589-96 e1.
2 Spergel J M, Brown-Whitehorn T F, Beausoleil J L, Franciosi J, Shuker M, Verma R, et al. 14 years of eosinophilic esophagitis: clinical features and prognosis. J Pediatr Gastroenterol Nutr 2009; 48:30-6.
3 Liacouras C A, Furuta G T, Hirano I, Atkins D, Attwood S E, Bonis P A, et al. Eosinophilic esophagitis: updated consensus recommendations for children and adults. J Allergy Clin Immunol 2011; 128:3-20 e6; quiz 1-2.
4 Alexander E S, Martin L J, Collins M H, Kottyan L C, Sucharew H, He H, et al. Twin and family studies reveal strong environmental and weaker genetic cues explaining heritability of eosinophilic esophagitis. J Allergy Clin Immunol 2014; 134:1084-92 e1.
5 Blanchard C, Wang N, Stringer K F, Mishra A, Fulkerson P C, Abonia J P, et al. Eotaxin-3 and a uniquely conserved gene-expression profile in eosinophilic esophagitis. J Clin Invest 2006; 116:536-47.
6 Kottyan L C, Davis B P, Sherrill J D, Liu K, Rochman M, Kaufman K, et al. Genome-wide association analysis of eosinophilic esophagitis provides insight into the tissue specificity of this allergic disease. Nat Genet 2014; 46:895-900.
7 Kottyan LC, Maddox A, Braxton JR, Stucke EM, Mukkada V, Putnam PE, et al. Genetic variants at the 16p13 locus confer risk for eosinophilic esophagitis. Genes Immun 2019; 20:281-92.
8 Rothenberg M E, Spergel J M, Sherrill J D, Annaiah K, Martin L J, Cianferoni A, et al. Common variants at 5q22 associate with pediatric eosinophilic esophagitis. Nat Genet 2010; 42:289-91.
9 Sleiman P M, Wang M L, Cianferoni A, Aceves S, Gonsalves N, Nadeau K, et al. GWAS identifies four novel eosinophilic esophagitis loci. Nat Commun 2014; 5:5593.
10 Price A L, Patterson N J, Plenge R M, Weinblatt M E, Shadick N A, Reich D. Principal components analysis corrects for stratification in genome-wide association studies. Nat Genet 2006; 38:904-9.
11 Purcell S, Neale B, Todd-Brown K, Thomas L, Ferreira M A, Bender D, et al. PLINK: a tool set for whole-genome association and population-based linkage analyses. Am J Hum Genet 2007; 81:559-75.
12 Das S, Forer L, Schonherr S, Sidore C, Locke A E, Kwong A, et al. Next-generation genotype imputation service and methods. Nat Genet 2016; 48:1284-7.
13 Kowalski M H, Qian H, Hou Z, Rosen J D, Tapia A L, Shan Y, et al. Use of >100,000 NHLBI Trans-Omics for Precision Medicine (TOPMed) Consortium whole genome sequences improves imputation quality and detection of rare variant associations in admixed African and Hispanic/Latino populations. PLoS Genet 2019; 15:e1008500.
14 Bulik-Sullivan B K, Loh P R, Finucane H K, Ripke S, Yang J, Schizophrenia Working Group of the Psychiatric Genomics C, et al. LD Score regression distinguishes confounding from polygenicity in genome-wide association studies. Nat Genet 2015; 47:291-5.
15 Watanabe K, Taskesen E, van Bochoven A, Posthuma D. Functional mapping and annotation of genetic associations with FUMA. Nat Commun 2017; 8:1826.
16 de Leeuw C A, Mooij J M, Heskes T, Posthuma D. MAGMA: generalized gene-set analysis of GWAS data. PLoS Comput Biol 2015; 11:e1004219.
17 Buniello A, MacArthur J A L, Cerezo M, Harris L W, Hayhurst J, Malangone C, et al. The NHGRI-EBI GWAS Catalog of published genome-wide association studies, targeted arrays and summary statistics 2019. Nucleic Acids Res 2019; 47:D1005-D12.
18 Ward L D, Kellis M. HaploReg v4: systematic mining of putative causal variants, cell types, regulators and target genes for human complex traits and disease. Nucleic Acids Res 2016; 44:D877-81.
19 Ferreira M A, Vonk J M, Baurecht H, Marenholz I, Tian C, Hoffman J D, et al. Shared genetic origin of asthma, hay fever and eczema elucidates allergic disease biology. Nat Genet 2017; 49:1752-7.
20 Xu H, Agalioti T, Zhao J, Steglich B, Wahib R, Vesely M CA, et al. The induction and function of the anti-inflammatory fate of TH17 cells. Nat Commun 2020; 11:3334.
21 Lo B C, Gold M J, Hughes M R, Antignano F, Valdez Y, Zaph C, et al. The orphan nuclear receptor ROR alpha and group 3 innate lymphoid cells drive fibrosis in a mouse model of Crohn's disease. Sci Immunol 2016;1.
22 Rajput C, Cui T, Han M, Lei J, Hinde J L, Wu Q, et al. RORalpha-dependent type 2 innate lymphoid cells are required and sufficient for mucous metaplasia in immature mice. Am J Physiol Lung Cell Mol Physiol 2017; 312:L983-L93.
23 Martinez-Martin N, Fernandez-Arenas E, Cemerski S, Delgado P, Turner M, Heuser J, et al. T cell receptor internalization from the immunological synapse is mediated by TC21 and RhoG GTPase-dependent phagocytosis. Immunity 2011; 35:208-22.
24 Martinez-Riano A, Bovolenta E R, Mendoza P, Oeste CL, Martin-Bermejo M J, Bovolenta P, et al. Antigen phagocytosis by B cells is required for a potent humoral response. EMBO Rep 2018; 19.
25 Vigorito E, Bell S, Hebeis B J, Reynolds H, McAdam S, Emson P C, et al. Immunological function in mice lacking the Rac-related GTPase RhoG. Mol Cell Biol 2004; 24:719-29.
26 Hunt K A, Zhernakova A, Turner G, Heap G A, Franke L, Bruinenberg M, et al. Newly identified genetic risk variants for celiac disease related to the immune response. Nat Genet 2008; 40:395-402.
27 Zhernakova A, Festen E M, Franke L, Trynka G, van Diemen C C, Monsuur A J, et al. Genetic analysis of innate immunity in Crohn's disease and ulcerative colitis identifies two susceptibility loci harboring CARDS and IL18RAP. Am J Hum Genet 2008; 82:1202-10.
28 Martino D, Neeland M, Dang T, Cobb J, Ellis J, Barnett A, et al. Epigenetic dysregulation of naive CD4+ T-cell activation genes in childhood food allergy. Nat Commun 2018; 9:3308.
29 Gerner M C, Ziegler L S, Schmidt R L J, Krenn M, Zimprich F, Uyanik-Unal K, et al. The TGF-b/SOX4 axis and ROS-driven autophagy co-mediate CD39 expression in regulatory T-cells. FASEB J 2020; 34:8367-84.
30 Komatsu N, Okamoto K, Sawa S, Nakashima T, Oh-hora M, Kodama T, et al. Pathogenic conversion of Foxp3+ T cells into TH17 cells in autoimmune arthritis. Nat Med 2014; 20:62-8.
31 Mehta A, Mann M, Zhao J L, Marinov G K, Majumdar D, Garcia-Flores Y, et al. The microRNA-212/132 cluster regulates B cell development by targeting Sox4. J Exp Med 2015; 212:1679-92.
32 Plotkin J B, Kudla G. Synonymous but not the same: the causes and consequences of codon bias. Nat Rev Genet 2011; 12:32-42.
33 Rauscher R, Ignatova Z. Timing during translation matters: synonymous mutations in human pathologies influence protein folding and function. Biochem Soc Trans 2018; 46:937-44.
34 Chi Y, Chai J, Xu C, Luo H, Zhang Q. The extracellular matrix protein matrilin-2 induces post-burn inflammatory responses as an endogenous danger signal. Inflamm Res 2015; 64:833-9.
35 Jonas A, Thiem S, Kuhlmann T, Wagener R, Aszodi A, Nowell C, et al. Axonally derived matrilin-2 induces proinflammatory responses that exacerbate autoimmune neuroinflammation. J Clin Invest 2014; 124:5042-56.
36 Wang L, Liu X, Song X, Dong L, Liu D. MiR-202-5p Promotes M2 Polarization in Allergic Rhinitis by Targeting MATN2. Int Arch Allergy Immunol 2019; 178:119-27.
37 Wang L, Yang X, Li W, Song X, Kang S. MiR-202-5p/MATN2 are associated with regulatory T-cells differentiation and function in allergic rhinitis. Hum Cell 2019; 32:411-7.
38 Fischer T A, Palmetshofer A, Gambaryan S, Butt E, Jassoy C, Walter U, et al. Activation of cGMP-dependent protein kinase Ibeta inhibits interleukin 2 release and proliferation of T cell receptor-stimulated human peripheral T cells. J Biol Chem 2001; 276:5967-74.
39 Franko A, Kovarova M, Feil S, Feil R, Wagner R, Heni M, et al. cGMP-dependent protein kinase I (cGKI) modulates human hepatic stellate cell activation. Metabolism 2018; 88:22-30.
40 Schmidtko A, Gao W, Konig P, Heine S, Motterlini R, Ruth P, et al. cGMP produced by NO-sensitive guanylyl cyclase essentially contributes to inflammatory and neuropathic pain by using targets different from cGMP-dependent protein kinase I. J Neurosci 2008; 28:8568-76.
41 Salas A, Hernandez-Rocha C, Duijvestein M, Faubion W, McGovern D, Vermeire S, et al. JAK-STAT pathway targeting for the treatment of inflammatory bowel disease. Nat Rev Gastroenterol Hepatol 2020; 17:323-37.
42 Ferreira M A R, Mathur R, Vonk J M, Szwajda A, Brumpton B, Granell R, et al. Genetic Architectures of Childhood- and Adult-Onset Asthma Are Partly Distinct. Am J Hum Genet 2019; 104:665-84.
43 Peterson K, Firszt R, Fang J, Wong J, Smith K R, Brady K A. Risk of Autoimmunity in EoE and Families: A Population-Based Cohort Study. Am J Gastroenterol 2016; 111:926-32.
44 Panda S K, Colonna M. Innate Lymphoid Cells in Mucosal Immunity. Front Immunol 2019; 10:861.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method for detecting a propensity for developing eosinophilic esophagitis (EoE) in a subject in need thereof, the method comprising: detecting in genotype information, the presence of at least one EoE associated genetic alteration in a target gene identified in said subject, the presence of said genetic alteration indicating said patient has an increased risk for developing eosinophilic esophagitis, wherein said genetic alteration is present in a gene sequence from one or more loci of TMEM182, RAD50, SOX4, MATN2, PRKG1, RHOG, SHANK2, GPR12, RORA, SMAD3, GALNT1, CPNE4, URGCP, NAMPT, JAK2, and/or CCNY, the method optionally comprising treating patients harboring said genetic alteration with an agent which ameliorates EoE and other autoimmune symptoms.

2. The method of claim 1, wherein said loci comprise single nucleotide polymorphisms that indicate that the genetic alteration is present, wherein the step of detecting the presence of said SNP comprises performing a process selected from the group consisting of detection of specific hybridization, measurement of allele size, restriction fragment length polymorphism analysis, allele-specific hybridization analysis, single base primer extension reaction, and sequencing of an amplified polynucleotide.

3. The method as claimed in claim 1, wherein in the target nucleic acid is DNA or RNA.

4. (canceled)

5. The method of claim 1, wherein nucleic acids genetic alteration are obtained from an isolated cell of the human subject.

6. The method as claimed in claim 1, further comprising an additional genetic alteration is present in a gene sequence from one or more loci of CAPN14, TSLP/WDR36, EMSY, and/or CLEC16A.

7. The method as claimed in claim 1, wherein the genetic alteration is a sex-specific alteration.

8. The method of claim 7, wherein the sex-specific alteration is at least one of TMEM182, CPNE4, and/or URGCP.

9. The method of claim 7, wherein the sex-specific alteration is at least one of NAMPT, JAK2, and/or CCNY.

10. The method as claimed in claim 1, wherein the subject suffers from at least one additional disease selected from asthma, allergies, atopic dermatitis, celiac disease, selective IgA deficiency, Systemic lupus erythematosus, multiple sclerosis, rheumatoid arthritis, inflammatory bowel disease, Chron's disease, ulcerative colitis, and/or type 1 diabetes.

11. A kit for practicing the method of claim 2.

12. A method for identifying agents which modulate eosinophilic esophagitis, comprising
a) providing cells expressing at least one nucleic acid comprising a genetic alteration as claimed in claim 1;
b) providing cells which express the cognate wild type sequence lacking said genetic alterations of step a);
c) contacting the cells of steps a) and b) with a test agent and
d) analyzing whether said agent alters a cellular parameter associated with the presence of eosinophilic esophagitis in the cells of step a) relative to those of step b), thereby identifying agents which alter said parameter.

13. The method of claim 12, wherein is said parameter is increased expression of IL-5 or IL-13.

14. The method of claim 12, wherein said cell is a blood cell or esophageal cell and said parameter is selected from the group consisting of epidermis development, epithelial cell differentiation, serine protease inhibition, altered cell cycle progression, or division, microtubule disruption, histone acetylation, DNA methylation, chromosomal segregation, ubiquitin conjugation, and phosphoinositide mediated signaling, and altered mitosis.

15. The method of claim 12, wherein said parameter is altered mRNA expression levels or altered protein expression levels of at least one gene selected from the group consisting of TMEM182, RAD50, SOX4, MATN2, PRKG1, RHOG, SHANK2, GPR12, RORA, SMAD3, GALNT1, CPNE4, URGCP, NAMPT, JAK2, and/or CCNY in blood cells or esophageal cells.

16. (canceled)

17. The method of claim 12, wherein said parameter is altered protein interactions between proteins encoded by at least one gene selected from the group consisting of TMEM182, RAD50, SOX4, MATN2, PRKG1, RHOG, SHANK2, GPR12, RORA, SMAD3, GALNT1, CPNE4, URGCP, NAMPT, JAK2, and/or CCNY and a protein binding partner in blood cells or esophageal cells.

18. The method of claim 12, wherein said parameter is altered signal transduction mediated by one or more proteins selected from the group consisting of TMEM182, RAD50, SOX4, MATN2, PRKG1, RHOG, SHANK2, GPR12, RORA, SMAD3, GALNT1, CPNE4, URGCP, NAMPT, JAK2, and/or CCNY.

19. (canceled)

20. The method of claim 1, further comprising administration of an agent for the treatment of EoE.

21. The method of claim 20, wherein said treatment ameliorates the symptoms of one or more of asthma, allergies, atopic dermatitis, celiac disease, selective IgA deficiency, Systemic lupus erythematosus, multiple sclerosis, rheumatoid arthritis, inflammatory bowel disease, Chron's disease, ulcerative colitis, and/or type 1 diabetes.

22. The method of claim 20, wherein said treatment is esophageal dilation, topical glucocorticoids, proton pump inhibitors, and corticosteroids.
